(19) United States
(12) Patent Application Publication
(10) Pub. No.: US 2006/0094121 A1
(43) Pub. Date: May 4, 2006

(54) Method for analysing amino acids, peptides and proteins

(75) Inventors: Gavin Reid, Royal Melbourne Hospital Victoria, MI (AU); Richard Simpson, Royal Melbourne Hospital Victoria (AU); Richard O'Hair, University of Melbourne (AU)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: Ludwig Institute For Cancer Research, New York, NY (US)

(21) Appl. No.: 10/535,389

(22) PCT Filed: Nov. 18, 2003
(86) PCT No.: PCT/US03/36739

§ 371 Date: Sep. 21, 2005

(30) Foreign Application Priority Data
Nov. 28, 2002 (AU) 2002952747

(51) Int. Cl. G01N 33/00

(52) U.S. Cl. 436/86

(57) ABSTRACT

The invention provides methods, reagents and kits for amino acid, peptide and protein identification, differential quantitation and for the analysis of post translational modification and cross-linking status, comprising: derivatizing a mixture of amino acids peptides or proteins, to form at least one amino acid peptide or protein containing a fixed-charge ion, other than at the C-terminal or N-terminal end thereof; introducing the mixture of amino acids peptides or proteins containing the fixed charge derivatized amino acid peptide or protein to a mass spectrometer; passing the mixture of amino acids peptides or proteins containing the fixed charge derivatized amino acid peptide or protein through a first mass resolving spectrometer to select precursor protein or peptide ions having a first desired mass-to-charge ratio; subjecting the precursor ions of the first mass to charge ratio to dissociation to form a product ion having a second mass-to-charge ratio that is characteristic of a fragmentation occurring at a site adjacent to the fixed charge; and detecting the product ions having the second mass-to-charge ratio. The product ion having the second mass-to-charge ratio may be either a product ion formed by neutral loss of the fixed charge from the precursor ion, or a product ion formed by charged loss of the fixed charge from the precursor ion.

### FIELD OF THE INVENTION

The present invention is concerned with a method for the analysis of amino acids, peptides or proteins.

### BACKGROUND OF THE INVENTION

With completion of the first draft of the human genome sequence, the challenge now facing researchers is to understand gene function. However, the biological function of a gene cannot be determined from a simple examination of its DNA sequence. Comprehensive analysis of the proteins expressed by the genome, therefore, promises to bridge the gap between the gene and its biological function. The term proteomics has become synonymous with (i) the identification and characterization of all proteins synthesized by a particular cell type or tissue at any given time, and quantitation of the global changes in protein expression levels observed between two different cell states (collectively known as expression proteomics) and, (ii) with the identification of components of functionally active protein complexes and characterization of the intricate protein-protein interactions involved in intracellular protein trafficking and signaling pathways (collectively known as cell-mapping proteomics). Taken together, these approaches allow comprehensive examination, at the protein level, of the complex cellular changes that occur following transformation of cells from one state to another [Blackstock, W. P. and Weir, M. P. Trends Biotechnol. 1999, 17, 121-127.; Pandey, A. and Mann, M. Nature 2000, 405, 837-846.].

Recent developments in mass spectrometry (MS), coupled with the development of sophisticated bioinformatic tools for database interrogation of MS derived data, have been the major factors enabling proteomics [Mann, M., Hendrickson, R. C. and Pandey, A. Annu. Rev. Biochem. 2001, 70, 437-473.]. In particular, the speed, specificity, and sensitivity of mass spectrometry make it especially attractive for use in strategies requiring rapid protein identification and characterization. Conventional MS approaches to proteomics generally involve one or two-dimensional electrophoretic (2DE) separation of protein mixtures, after which the protein spot or gel slice is cut out and subjected to in situ proteolysis using trypsin. Peptides are then extracted and subjected to mass spectrometric analysis. The masses of these peptides are characteristic of the protein, and provide a peptide “mass fingerprint” which can be used in database searches to identify the protein [Henzel, W. J. Billeci T. M., Stults J. T., Wong S. C., Grimley C. and Watanabe C. Proc. Natl. Acad. Sci. USA. 1993, 90, 5011-5015.]. A more comprehensive approach, particularly for the identification and quantitation of individual components present in complex protein mixtures, is to subject each of the proteolytically derived peptides to tandem mass spectrometry. Subsequent identification of each peptide may be performed by either database analysis of the uninterpreted product ion spectrum [Eng, J. K. McCormack, A. L. and Yates, J. R. J. Am. Soc. Mass Spectrom. 1994, 5, 976-989.], through database searching of a partially derived amino acid “sequence tag” [Mann, M. and Wilm, M. Anal. Chem. 1994, 66, 4390-4399.], or by “de-novo” sequence analysis [Hunt, D. F., Yates, J. R., Shabanowitz J., Winston S. and Hauer, C. R. Proc. Natl. Acad. Sci. USA 1986, 83, 6233-6237.].

This general analysis strategy is complicated however, by several limitations.

Foremost is that 2D-gels are only capable of resolving approximately 1500-2000 proteins, yet there are typically greater than 8000 proteins expressed per cell. Therefore, only the most abundant proteins are observed and a significant portion of 2-DE separated gel spots contain more than one protein due to co-electrophoresis and/or differentially modified (or processed) forms of the same protein. Additionally, several classes of proteins, notably hydrophobic proteins, low abundance proteins, and those with extremes of pI and molecular weight are poorly represented in 2D-gel based separations [Gygi, S. P., Corthals, G. L., Zhang, Y., Rochon, Y., Aebersold, R. Proc. Natl. Acad. Sci. U.S.A. 2000, 97, 9390-9395.]. Also, 2-DE is labor-intensive, slow, technically demanding, often suffers from poor reproducibility, and hence is not readily amenable to high-throughput or automation.

Secondly, while the identification of only one peptide from a protein digest by MS/MS is required to unambiguously identify that protein, provided that the peptide is unique to a single protein, in practice a significant amount of time during the analysis of complex peptide mixtures is spent in analyzing either the same peptide ion (e.g., in sequential scans during chromatographic separation of a complex peptide mixture) or different peptides of the same protein. Routinely therefore, peptides present at high relative abundance are preferentially sampled, and information regarding the identity of proteins represented in the complex mixture as low abundance peptides is commonly not obtained. Also, up to a quarter of peptide MS/MS spectra obtained during MS based peptide sequencing are unassignable (i.e., product ion spectra are too complex, too low in abundance, or lack sufficient ions to enable their interpretation) [Simpson, R. J., Connelly, L. M., Eddes, J. S., Pereira, J. J., Moritz, R. L. and Reid, G. E. Electrophoresis. 2000, 21, 1707-1732].

In order to partially overcome these limitations, dynamic exclusion during MS/MS acquisition has been employed to allow greater numbers of distinct peptide ions to be selected throughout the course of an LC/MS/MS experiment [Davis, M. T.; Spahr, C. S.; McGinley, M. D.; Robinson, J. H.; Bures, E. J.; Beierle, J.; Mort, J.; Yu, W.; Luethy, R; Patterson, S. D. Proteomics 2001, 1, 108-117.]. Several strategies for simplification of the peptide mixture prior to mass spectrometric analysis, either by multidimensional chromatographic methods [Washburn, M. P., Wolters, D. and Yates, J. R. Nat. Biotechnol. 2001, 19, 242-247.], or by selective enrichment in solution of only those peptides containing certain amino acids, by affinity selection [Spahr, C. S. Susin, S. A., Bures, E. J., Robinson, J. H., Davis, M. T., McGinley, M. D., Kroemer, G. and Scott D. Patterson, S. D. Electrophoresis. 2000, 21, 1635-1650] or by differential chromatography [Gevaert, K., Van Damme, J., Goethals, M., Thomas, G. R., Hoorelbeke, B., Demol, H., Martens, L., Puype, M., Staes, A. and Vandekerckhove, J. Mol. Cell. Proteomics. 2002, 1, 896-903.], have also been described. In combination with the dynamic exclusion and multidimensional chromatographic approached outlined above, these methods allow further increases in the number of proteins that can be identified by mass spectrometric analysis. However, the identification of low abundance proteins in these complex mixtures, where the expression of individual proteins may vary from 10 to 10⁶ copies per cell, including those potentially containing important information such as the structure and location of post-translational modifications, still remains a significant challenge.

A fundamental aspect of proteomics research is the determination of protein expression levels between two different states of a biological system (i.e., relative quantification of protein levels), such as that encountered between a normal and diseased cell or tissue. As there is a marked disparity between changes in mRNA expression levels (transcriptomics) and their corresponding proteins (proteomics), it is clear that array-based gene expression monitoring or other gene expression methods for measuring mRNA abundances, alone, are insufficient for analyzing the cell's protein complement [Gygi, S. P., Rochon, Y., Franza, B. R. and Aebersold, R. Mol. Cell. Biol. 1999, 19, 1720-1730.].

Qualitative and quantitative analysis of changes in protein expression profiles as a function of, for example, cell-cycle regulation, disease state or drug exposure has traditionally been performed by 2DE via image analysis of individual stained protein spots, due to the high dimensionality afforded by the orthogonal isoelectric point and molecular weight separation modes [Patton & Beechem Curr Opinion Chem Biol 2002, 6, 63-69.; Zhou, G., Li, H., DeCamp, D., Chen, S., Shu, H., Gong, Y., Flaig, M., Gillespie, J. W., Hu, N., Taylor, P. R., Emmert-Buck, M. R., Liotta, L. A., Petricoin, E. F. and Zhao, Y. Mol Cell Proteomics 2002 1, 117-123]. However, these 2DE gel based quantitation approaches suffer from the same limitations as those discussed above for 2DE gel based protein identification, thereby limiting quantitative analysis to those proteins that are present as relatively abundant, pure spots on the gel and that can be adequately visualized by the staining method employed [Gygi, S. P., Corthals, G. L., Zhang, Y., Rochon, Y., Aebersold, R. Proc. Natl. Acad. Sci. U.S.A. 2000, 97, 9390-9395.]. Some of these shortcomings have been overcome by the use of more sensitive detection methods, such as the SYPRO ruby fluorescent protein stain [Berggren, K., Chemokalskaya, E., Steinberg, T. H., Kemper, C., Lopez, M. F., Diwu, Z., Haugland, R. P. and Patton, W. F. Electrophoresis 2000, 21, 2509-2521], and by the use of 2-D difference gel electrophoresis (DIGE) [Unlu, M., Morgan, M. E. and Minden, J. S. Electrophoresis, 1997, 18, 2071-2077.]. However, these methods are fundamentally reliant on the use of adequate software packages for spot detection, gel matching and spot quantitation [Raman, B., Cheung, A. and Martin, M. R. Electrophoresis, 2002, 23, 2194-2202.] and cannot account for situations where overlapping protein spots are present.

Two general approaches for MS based quantitation of differential protein expression levels between two different cell/tissue states, using isotopic labeling, have been developed. First, the groups of Smith [Pasa-Tolic, L; Jensen, P. K.; Anderson, G. A.; Lipton, M. S.; Peden, K. K.; Martinovic, S; Tolic, N; Bruce, J. E.; Smith, R. D. J. Am. Chem. Soc. 1999, 121, 7949-7950.; Veenstra, T. D., Martinovic, S., Anderson, G. A., Pasa-Tolic, L. and Smith, R. D. J. Am. Soc. Mass Spectrom. 2000, 11, 78-82.; Conrads, T. P. et. al. Anal. Chem. 2001, 73, 2132-2139; Smith, R. D.; Anderson, G. A.; Lipton, M. S.; Pasa-Tolic, L.; Shen, Y.; Conrads, T. P.; Veenstra, T. D.; Udseth, H. R. Proteomics 2002,2, 513-523.], Chait [Oda, Y.; Huang, K.; Cross, F. R.; Cowburn, D.; Chait, B. T. Proc. Natl. Acad. Sci. U.S.A. 1999, 96, 6591-6596.], and others [Chen, X., Smith, L. M., Bradbury, E. M. Anal. Chem. 2001, 72, 1134-1143; Ong, Shao-En., Blagoev, B., Kratchmarova, I., Bach-Kristensen, D., Steen, H., Pandey, A. and Mann, M. Mol. Cell. Proteomics. 2002, 1, 376-386; Jiang, H. and English, A. M. J. Proteome. Res. 2002, 1, 345-350; Zhu, H., Hunter, T. C., Pan, S., Yau, P. M., Bradbury, E. M. and Chen, X. Anal. Chem. 2002, 74, 1687-1694.] have all separately presented results involving the metabolic incorporation of isotopically depleted (¹³C-, ¹⁵N-, and ²H-depleted) or enriched amino acids (either by uniform labeling with ¹⁵N, or by incorporation of selected amino acids containing heavy isotopes (eg., ¹³C, ¹⁵N, ²H)) into a cellular protein population. After isolation of proteins from the cellular matrix, the sample is combined with one incorporating natural isotopes, then individual proteins are resolved by electrophoretic or chromatographic methods, digested and the masses of the peptides determined by MS. The site specific labels allow efficient identification of those peptides containing the enriched amino acid mass “tags”, via comparison with their unlabelled forms. Additionally, by comparing the relative abundances of the peptides from the isotopically enriched sample with those from a sample prepared using naturally abundant isotopes, quantitation of changes in the level of protein expression between the two samples may be obtained. This in vivo labelling approach is limited, however, to those systems where cells can be cultured under conditions suitable for incorporation of the isotopic label.

The second approach involves in vitro chemical derivatization with isotopically enriched labels following isolation of the proteins from the cellular matrix. One approach that has received much attention to date is the isotope coded affinity tag (ICAT) technique developed by Aebersold and co-workers [Gygi, S. P.; Rist, B; Gerber, S. A.; Turecek, F; Gelb, M. H.; Aebersold, R. Nat. Biotechitol. 1999, 17, 994-999.; Smolka, M; Zhou, H; Aebersold, R. Mol. Cell. Proteomics. 2002, 1, 19-29.; Shiio, Y; Donohoe, S; Yi, E. C.; Goodlett, D. R.; Aebersold, R; Eisenman, R. N. EMBO 2002, 21, 5088-5096.; Han, D. K., Eng, J., Zhou, H. and Aebersold, R. Nature Biotechnol. 2001, 19, 946-951.; Griffin, T. J., Han, D. K. M., Gygi, S. P., Rist, B., Lee, H., Aebersold, R. and Parker, K. C. J. Am. Soc. Mass. Spectrom. 2001, 12, 1238-1246.; Gygi, S. P., Rist, B. and Aebersold, R. Current Opinions in Biotechnol. 2000, 11, 396-401.]. Proteins from two different cell/tissue states are reduced and S-alkylated with either naturally abundant (light) or isotopically enriched (heavy) ICAT reagents, respectively, each containing a biotin moiety for subsequent affinity selection of cysteine-containing peptides by streptavidin affinity purification, leading to simplification of the mixture prior to MS analysis. In the same manner as described above for metabolic labelled samples, the abundance ratios of the proteolytically derived peptides containing the heavy isotope “diseased” sample compared to those originating from the light isotope “normal” sample (or between those containing structural labels) for each of these methods are indicative of changes in the level of protein expression between the two samples, thereby allowing their differential quantitation.

A number of similar strategies for selective peptide identification and differential quantitation have since been described [Spahr, C. S. Susin, S. A., Bures, E. J., Robinson, J. H., Davis, M. T., McGinley, M. D., Kroemer, G. and Patterson, S. D. Electrophoresis. 2000, 21, 1635-1650.; Adamczyk, M. and Gebler, J. C.; Wu, J. Rapid. Commun. Mass Spectrom. 1999, 13, 1813-1817.; Sechi, S. Anal. Chem. 1998, 70, 5750-5158.; Sechi, Salvatore. Rapid Commun. Mass Spectrom. 2002, 16, 1416-1424.; Gehanne, S., Cecconi, D., Carboni, L., Giorgio Righetti, P., Domenici, E. and Handam, M. Rapid Commun. Mass Spectrom. 2002, 16, 1692-1698.; Wang, S.; Regnier, F. E. J Chrom. A 2001, 924(1-2), 345-357.; Amini, A; Chakraborty, A; Regnier, Fred E. J. Chrom. B. 2002,772, 35-44.; Wang, S., Zhang X. and Regnier, F. E. J. Chrom. A. 2002, 949, 153-162.; Mirgorodskaya, O. A. Kozinin, Y. P., Titov, M. L, Korner, R., Sonksen, C. P. and Roepstorff, P. Rapid Commun. Mass Spectrom. 2000, 14, 1226-1232.; Stewart, I. I.; Thomson, T; Figeys, D. Rapid Commun. Mass Spectrom. 2001, 15, 2456-2465.; Yao, X.; Freas, A; Ramirez, J.; Demirev, P. A.; Fenselau, C. Anal. Clem. 2001, 73, 2836-2842.; Peters, E. C., Horn, E. C., Tully, D. C. and Brock, A. Rapid Commun. Mass Spectrom. 2001, 15, 2387-2392.; Muenchbach, M., Quadroni, M., Miotto, G. and James, P. Anal. Chem. 2000, 72,4047-4057; Chakraborty, A; Regnier, F. E. J. Chrom. A 2002, 949, 173-184.; Geng, M.; Ji, J.; Regnier, F. E. J. Chrom. A 2000, 870, 295-313.; Ji, J.; Chakraborty, A.; Geng, M.; Zhang, X.; Amini, A.; Bina, M.; Regnier, F. J. Chrom. B 2000, 745,197-210.; Goodlett, D. R., Keller, A., Watts, J. D., Newitt, R., Yi, E. C., Purvine, S., Eng, J., von Haller, P., Aebersold, R. and Kolker, E. Rapid Commun. Mass Spectrom. 2001, 15, 1214-1221.; Liu, P; Regnier, F. E. J. Proteome Res. 2002, 1,443-450.]. More recently, two groups have described covalent solid phase cysteine capture methods for mixture simplification. [Zhou, H.; Ranish, J. A.; Watts, J. D.; Aebersold, R. Nature Biotechnol. 2002, 20, 512-515.; Qiu, Y; Sousa, E. A.; Hewick, R. M.; Wang, J. H. Anal. Chem. 2002, 74, 4969-4979.]. Several non-isotopic labelling strategies employing structural labelling [Cagney, G., Emili, A. Nature Biotechnol. 2002, 20, 163-170.; Beardsley, R. L. and Reilly, J. P. J. Proteome Res. 2003, 2, 15-21.], as well as a method for performing differential quantitation by comparing the chromatographically resolved MS ion abundances between a sample of interest and a control have also been described [Bondarenko, P. V., Chelius, D. and Shaler, T. A. Anal. Chem. 2002, 74, 4741-4749.; Chelius, D. Bondarenko. P. V. J. Proteome Res. 2002, 1, 317-323.]. A number of reviews have compared the relative merits and limitations of several of these approaches [Moseley, M. A. Trends in Biotechnol. 2001, 19, S10-S16.; Patton, W. F., Schulenberg, B. and Steinberg, T. H. Curr. Opin. Biotechnol. 2002, 13, 321-328.; Turecek, F. J. Mass Spectrom. 2002, 37, 1-14.; Regnier, F. E.; Riggs, L.; Zhang, R.; Xiong, L.; Liu, P.; Chakraborty, A.; Seeley, E.; Sioma, C.; Thompson, R. A. J. Mass Spectrom. 2002, 37,133-145.].

The two major post translational modifications (PTM's) of proteins are phosphorylation and glycosylation. Of these, the reversible phosphorylation of proteins ranks among the most important PTM that occurs in the cell. Commonly, two approaches for enrichment of phosphoprotein/phosphopeptides prior to their detection and subsequent microcharacterisation have been employed. There are (i) Immunoprecipitation; [Gronborg, M., Kristiansen, T. Z., Stensballe, A., Andersen, J. S., Ohara, O., Mann, M., Jensen, O. N. and Pandey, A. Mol. Cell. Proteomics. 2002, 1, 517-527.], and (ii) Immobilized metal ion affinity chromatography (IMAC); [Posewitz, M. C. and Tempst, P. Anal. Chem. 1999, 71, 2883-2892.; Ficarro, Scott B.; McCleland, Mark L.; Stukenberg, P. Todd; Burke, Daniel J.; Ross, Mark M.; Shabanowitz, Jeffrey; Hunt, Donald F.; White, Forest M. Nat Biotechnol. 2002, 20, 301-305.].

Proteins resolved by 2D gels or 1D SDS-PAGE can be detected by autoradiography or storage phosphorimaging using in vivo or in vitro ³²P labelling [Ji H, Baldwin G S, Burgess A W, Moritz R L, Ward L D, and Simpson R J. J Biol Chem 1993, 268, 13396-13405.; Boyle W. J., Geer Van der P. and Hunter T. Methods Enzymol. 1991, 201, 110-149.; Yan J. X., Packer N. H., Gooley A. A. and Williams K. L. J. Chromatogr. 1998, 808, 23-41.], or by western blotting using antibodies to detect phosphorylated proteins. Historically, sequencing of ³²P radiolabelled peptides was performed by Edman degradation [Wettenhall, R. E., Aebersold, R. H., and Hood, L. E. Methods Enzymol 1991, 201, 186-99.]. However, this approach typically requires prior knowledge of the protein sequence in order to correlate a loss of radioactivity, indicative of the phosphorylation site, with the amino acid sequence of the peptide and can be hampered by a low stoichiometry of phosphorylation at a given site [Katze, M. G., Kwieciszewski, B., Goodlett, D. R., Blakely, C. M., Nedderman, P., Tan, S-L, Aebersold, R. Virology. 2000, 278, 501-513], or by the presence of multiple differentially phosphorylated forms of the same protein being present [Storm, S. M., and Khawaja, X. Z. Brain. Res. Mol. Brain. Res. 1999, 71, 50-60.].

More recently, mass spectrometry has proven to be particularly useful for the analysis of protein phosphorylation [Neubauer, G. and Mann, M. Anal Chem 1999, 71, 235-242.]. However, the mass spectrometric analysis of phosphopeptides is more complicated compared to that for unmodified peptides, due to their lower ionization efficiencies in positive ion MS analysis mode [Jiao P. C., Teykam J., Andrews P. C., Cage D. A. and Allison J. Anal. Biochem. 1994., 219, 9-20.], and the difficulties encountered in rapidly switching between ionisation polarities, to allow both identification (in negative mode) and characterization (in positive mode), during the course of a single experiment [Janek K., Wenschuh H., Bienert M., and Krause E. Rapid Commwn. Mass Spectrom. 2001, 15, 1593-1599.; Ma Y., Lu Y., Mo W., and Neubert T. A. Rapid Commun. Mass Spectrom. 2001, 15, 1693-1700.]. Also, phosphoserine- and phosphothreonine-containing peptides can readily undergo the facile loss of phosphoric acid (H₃PO₄) upon ESI and MALDI ionization, and upon low energy CID.

There are several ways to circumvent the difficulties associated with ionization suppression of phosphopeptides in positive ion mode. The sample may be enriched for phosphopeptides, as discussed above, in order to reduce the excess of unmodified peptides that suppress ionisation. However, regardless of attempts made to overcome signal suppression in the analysis of phosphopeptides, limitations still exist when attempting to identify the site of phosphorylation by tandem mass spectrometry, due to the lability of the phosphate side chain. A simple solution is to perform analysis of the phosphopeptide samples by the parallel analysis of samples treated with and without alkaline phosphatase [Larsen, M. R., Sorensen, G. L., Fey, S. J., Larsen, P. M. and Roepstorff, P. Proteomics, 2001, 1, 223-238.]. Phosphopeptide identification may also be performed by precursor ion scan mode monitoring of the characteristic phosphate specific product ions at m/z 79 (PO₃), and m/z 89 (H₂PO₄), following collision induced dissociation (CID) of the phosphopeptide ions in negative ion mode, or by monitoring for the loss of H₃PO₄ (98 Da) or HPO3 (80 Da) in positive ion neutral loss scan mode [Carr S. A., Huddleston M. J., and Annan R. S. Anal. Biochem. 1996,239, 180-192.; Schlosser A., Pipkom R., Bossemeyer D., and Lehmann W. D. Anal. Chem. 2001, 73,170-176.].

Product ion scan mode MS/MS of the intact phosphopeptide can sometimes be used to characterize the precise site of phosphate attachment within the peptide, as the b- and y-type fragment ions formed from peptide backbone cleavage may contain information on the specific location of the phosphorylated amino acid residue. However, as indicated above, this technique is often hampered by extensive gas-phase dephosphorylation and elimination of phosphoric acid (H₃PO₄, 98 Da) from phosphoserine- and phosphothreonine, making it difficult to unambiguously locate the site of modification. The pbosphotyrosine side chain, however, is relatively stable under MS and MS/MS conditions due to the relatively high stability of the arylphosphate modification, therefore the location of phosphotyrosine residues can be readily determined by the mass difference benveen two successive fragment ions of 243 Da. Indeed, the characteristic ‘reporter’ immonium ion of phosphotyrosine at 216.043 Da has been used for precursor ion experiments in positive ion mode MS/MS for selective identification of phosphotyrosine containing peptides [Steen H., Kuster B., Fernandez M., Pandey A., and Mann M. 2001. Anal. Chem. 73: 1440-1448].

A common way to overcome the lability of the phosphoserine and phosphothreonine side chains during MS/MS is to replace the phosphate group with a more stable, less acidic side chain functionality. This can be accomplished by elimination of phosphoserine and phosphothreonine residues under strongly alkaline conditions to yield dehydroalanine or dehydroaminobutyric acid residues, respectively. Subsequent Michael addition of a nucleophile allows a simple means for derivatizing the formerly phosphorylated serine or threonine residues prior to mass spectrometric analysis [Weckwerth, W., Willmitzer, L. and Fiehn, O. Rapid Commun. Mass Spectrom. 2000,14, 1677-1681.; Molloy, M. P. and Andrews, P. C. Anal Chem. 2001, 73, 5387-5394.; Li, W., Boykins, R. A., Backlund, P. S., Wang, G. and Chen, H-C. Anal Chem. 2002 74,5701-5710.; Steen, H. and Mann, M. J. Am. Soc. Mass Spectrom. 2002, 13, 996-1003.]. The method can also be used to incorporate an affinity “tag”, thereby allowing enrichment of the derivatized peptides prior to their analysis [Adamczyk, M., Gebler, J. C. and Wu, J. Rapid Commun Mass Spectrom. 2001, 15, 1481-1488.; Oda, Y., Nagasu, T. and Chait, B. T. Nature Biotechnol. 2001, 19, 379-382.], and, when coupled with the incorporation of an isotopic label, for quantitation of the degree of phosphorylation observed between two different sample sets [Goshe M. B., Conrads, T. P., Panisko, E. A., Angell, N. H., Veenstra, T. D. and Smith, R. D. Anal Chem. 2001, 73, 2578-2586.; Goshe, M. B., Veenstra, T. D., Panisko, E. A., Conrads, T. P., Angell, N. H. and Smith, R. D. Anal. Chem, 2002, 74, 607-616.; Adamczyk, M; Gebler, J. C. and Wu, J. Rapid Commun. Mass Spectrom. 2002, 16, 999-1001.].

Note that β-elimination affects both O-phospho and O-glycosidic linkages to the same extent. Therefore the β-elimination method cannot distinguish between O-linked post translational modifications of serine and threonine residues.

Another chemical method for analyzing the phosphoproteome, which is applicable to phosphotyrosine-containing peptides as well as those containing phosphoserine and phosphothreonine residues, has recently been described [Zhou, H., Watts, J. D. and Aebersold, R. Nature Biotechnol 2001, 19, 375-378]. For a recent review of quantitative phosphoproteome analysis see [Mann, M., Ong, S-E., Gronborg, M., Steen, H., Jensen, O. N. and Pandey, A. Trends Biotechnol. 2002, 20, 261-268.].

Determination of protein-protein interactions are a crucial aspect of integrated biological studies aimed at understanding the complex pathways involved in cellular signaling and protein trafficking. Already, a large proportion of known protein-protein interactions in yeast [Bader, G. D. and Hogue, C. W. V. Nature Biotechnol. 2002, 20, 991-997] have been identified by genome-scale yeast two-hybrid assays [Uetz, P. et al. Nature 2000, 403, 623-627.; Ito, T., Chiba, T., Ozawa, R., Yoshida, M., Hattori, M. and Sakaki, Y. Proc. Natl. Acad. Sci. USA, 2001, 98, 4569-4574.; Legrain, P. Nature. Biotechnol. 2002, 20, 128-129.], and direct affinity capture methods such as (i) co-precipitation using affinity-tagged recombinant proteins [Gavin, A-C. et al. Nature 2002, 415, 141-147.; Ho, Y. et al. Nature. 2002, 415, 180-183.], (ii) ‘pull down’ techniques using antibodies directed against one of the component proteins, (iii) protein-affinity-interaction chromatography (e.g., using recombinant glutathione S-transferase (G ST)-fusion proteins and glutathione-affinity chromatography), or (iv) isolation of intact multiprotein complexes (e.g., nuclear pore complexes, ribosome complexes, and spliceosomes). These later methods, coupled with mass spectrometry for protein identification, constitute the major methods for cell mapping proteomics, which aims to describe all protein-protein interactions (both spatially and temporally) within a given cell [Blackstock, W. P. and Weir, M. P. Trends Biotechnol. 1999, 17, 121-127.]. However, the interactions detected by these physical methods may include large numbers of non-specific interactions with no biological significance [Bader, G. D. and Hogue, C. W. V. Nature Biotechinol. 2002, 20, 991-997.].

Mass spectrometry combined with cross-linking [Rappsilber, J., Siniossoglou, S., Hurt, E. C., and Mann, M. Anal. Chem. 2000, 72, 267-275.], or hydrogen/deuterium exchange [Yamada, N., Suzuki, E., and Hirayama, K Rapid Commun. Mass Spectrom. 2002, 16, 293-299] can be used for the rapid low-resolution evaluation of the three dimensional structures of proteins and protein complexes. Cross-linking generally involves chemical [Uy, R., and Wold, F., 1977, In: “Protein Cross-linking” (Friedman. M., ed.), Plenum, New York.; Fancy, D. A. Current Opin. Chlem. Biol. 2000, 4, 28-33.] or photochemical [Chowdhry, V., and Westheimer, F. H., Annu. Rev. Biochem. 1979, 48, 293-325.; Fancy, D. A. and Kodadek, T. Proc. Natl. Acad. Sci. USA 1999, 96, 6020-6024.] cross-linking of an isolated protein complex, followed by proteolytic digestion and MS and/or MS/MS analysis of the resulting peptide mixture, to subsequently locate proximally adjacent regions of the proteins being examined. However, the large number of peptide species that are generated following digestion of cross-linked proteins makes it often difficult to rapidly and unambiguously identify those peptides that are cross-linked. A number of different groups have developed methods to partially overcome this limitation, either by prior reduction of thiol cleavable cross-linkers [Bennett, K. L., Kussmann, M., Bjork, P., Godzwon, M., Mikkelsen, M., Sorensen, P. and Roepstorff, P. Protein Sci. 2000, 9, 1503-1518.], by using isotopic labelling methodologies [Chen, X., Chen, Y. H., and Anderson, V. E. Anal. Biochem 1999, 273, 192-203.; Pearson, K. M., Panell, L. K. and Fales, H. M. Rapid Commun. Mass Spectrom. 2002, 16, 149-159.; Muller, D. R., Schindler, P., Towbin, H., Wirth, U., Voshol, H., Hoving, S., and Steinmetz, M. O. Anal. Chem. 2001, 73, 1927-1934.; Back, J. W., Notenboom, V., de Koning, L. J., Muijsers, A. O., Sixma, T. K., de Koster, C. G., and de Jong, L. Anal. Chem. 2002, 74, 4417-4422.; Taverner, T., Hall, N. E., O'Hair, R. A. J. and Simpson, R. J. J. Biol. Cliem. 2002, 277, 46487-46492], or via the incorporation of a labile MS/MS “tag” on the cross linker itself [Back, J. W., Hartog, A. F., Dekker, H. L., Muijsers, A. O., de Koning, L. J. and de Jong, L. J Am. Soc. Mass Spectrom. 2001, 12, 222-227.].

It is important to recognise that all of the techniques described above for selective identification and differential quantitation based on incorporation of a differential isotopic “signature” rely on a common approach for peptide identification. That is, the isotopic signatures of the peptide ions of interest are detected by mass analysis of their intact precursor ions. Thus, limitations of the approach are encountered when; (i) ions of interest are present at low levels (i.e., approaching or below the level of chemical noise present in the spectrum) [Knitchinsky, A. N. and Chait, B. T. J. Am. Soc. Mass Spectrom. 2002, 13, 129-134.], (ii) the masses of low abundance differentially labeled peptides overlap with other higher abundance components present in the mixture, (iii) separation of the differential labelled “heavy” and “light” peptides occurs during chromatographic fractionation of the peptide mixture [Zhang, R.; Sioma, C. S.; Wang, S.; Regnier, F. E. Anal. Chem. 2001, 73, 5142-5149. Zhang, R., Sioma, C. S.; Thompson, R. A.; Xiong, L.; Regnier, F. E. Anal. Chem. 2002, 74, 3662-3669.; Zhang, R. and Regnier, F. E. J. Proteome Res. 2002, 1, 139-147], or (iv) the mass spectrometer lacks sufficient resolution to adequately resolve the two labelled components, thereby precluding their detection.

Tandem mass spectrometry (S/MS) dissociation methods [McLuckey, S. A. and Goeringer, D.E. J. Mass Spectrom. 1997, 32, 461-474.] methods, whereby a precursor ion of interest is mass selected, subjected to fragmentation via collision-induced dissociation (CID) and then the resultant product ions mass analyzed to derive structural information relating to the amino acid sequence of the peptide or to indicate the presence and location of post-translational modifications, may be used to address at least some of the limitations indicated above. Due to the reduction in chemical noise associated with the formation of product ions at different m/z values to that of the mass selected precursor ion, low abundance ions that may not otherwise be apparent in the mass spectrum (i.e., below the level of chemical noise) may be successfully detected and analysed by MS/MS methods. Indeed, operation of a tandem mass spectrometer in precursor ion [Schwartz, J. C.; Wade, A. P.; Enke, C. G.; Cooks, R. G. Anal. Chem. 1990, 62, 1809-1818.; Schlosser A., Pipkorn R., Bossemeyer D., and Lehmann W. D. 2001. Anal. Chem. 73: 170-176.; McClellan, J. E.; Quarnby, S. T.; Yost, R. A.; Anal. Chem. 2002, 74, 5799-5806.] or neutral loss [Schwartz, J. C.; Wade, A. P.; Enke, C. G.; Cooks, R. G. Anal. Chem. 1990, 62, 1809-1818.; Wihn M; Neubauer G; Mann M. Anal. Chem. 1996, 68, 527-33.; McClellan, J. E.; Quarnby, S. T.; Yost, R. A.; Anal. Chem. 2002, 74, 5799-5806.) MS/MS scan modes, whereby the mass spectrometer is set to detect diagnostic low mass product ions, or product ions offset by a given mass from the precursor ion, respectively, has been demonstrated to increase sensitivity by 1-2 orders of magnitude over conventional MS based detection methods [Wilm M; Neubauer G; Mann M. Anal. Chem. 1996, 68, 527-533.]. These precursor ion or neutral loss scan modes can be used to indicate the presence of peptides containing certain structural features via formation of diagnostic “signature ions” formed upon CID MS/MS, giving greater specificity compared to that of MS based methods, and overcoming the problem of low abundance peptides overlapping with other higher abundance components present in the mixture. One of the limitations of MS/MS based approaches however, is that fragmentation giving rise to the product ion or neutral loss of interest is usually only one of many dissociation channels, thereby “diluting” the spectrum, limiting sensitivity and potentially complicating subsequent interpretation of the spectra. Also, MS/MS methods for quantitation of differential protein expression have not been described.

### SUMMARY OF THE INVENTION

We have developed a novel strategy for protein identification, differential quantitation and for the analysis of post translational modification status and cross-linking, involving the fixed-charge derivatization of selected amino acids, peptides or proteins containing certain structural features, (e.g., the side chains of selected amino acids, or those containing post translational modifications or cross linking agents), that is, in its preferred forms, capable of addressing all of the MS and MS/MS limitations discussed above, with an expected increase in selectively and sensitivity of several orders of magnitude over the existing MS based approaches.

Derivatization strategies for mass spectrometric analysis are commonplace and have been reviewed previously [Knapp, D. R. Methiods Enzymology 1990, 193, 314-329.; Anderegg, R. J. Mass Spectroin. Rev. 1988, 7, 395-424.; Roth, K. D. W., Huang, Z-H., Sadagopan N, and Watson J. T. Mass Spectrom. Rev. 1998, 17, 255-274.; Sadagopan, N. and Watson J. T. J. Am. Soc. Mass. Spectrom. 2001, 12, 399-409.; Jones, M. B., Jeffrey, W. A., Hansen, H. F., Pappin, D. J. C. Rapid Commun. Mass Spectrom. 1994, 8, 737-42.; Spengler, B., Luetzenkirchen, F., Metzger, S., Chaurand, P., Kaufinann, R., Jeffery, W., Bartlet-Jones, M. and Pappin, D. J. C. Int. J Mass Spectrom. Ion Proc. 1997, 169/170, 127-140.; Keogh, T., Lacey, M. P., and Youngquist, R. S. Rapid. Commum. Mass Spectrom. 2000, 14, 2348.]. Although fixed charge derivatives of peptides have been used in conjunction with tandem mass spectrometry for sequencing applications, previous work has entirely focused on directing fragmentation toward the formation of a particular series of backbone cleavage derived sequence ions (i.e., maximizing sequence coverage), and has been limited to the derivatization of the N- and C-termini, as well as lysine and arginine side chains.

In contrast, the present invention is based on a fixed-charge derivatization approach, which is designed to direct the dissociation of the peptide toward a single predictable fragmentation channel, resulting in the formation of a single product, thereby allowing its selective identification from a complex mixture by precursor ion or neutral loss scan mode MS/MS, then subjecting it to fer structural interrogation, using MS/MS or multistage MS/MS (MSn), or by determination of an “accurate mass tag” of the precursor or product ion [Conrads, T. P., Anderson, G. A., Veenstra, T. D., Pasa-Tolic, L. and Smith, R. D. Anal. Chem. 2000, 72, 3349-3354.; Goodlet, D. R., Bruce, J. E., Anderson, G. A., Rist, B., Pasa-Tolic, L., Fiehn, O., Smith, R. D. and Aebersold, R. Anal. Chem. 2000, 72, 1112-1118.; Strittinatter, E. F., Ferguson, P. L., Tang, K. and Smith, R. D. J. Am. Soc. Mass Spectrom. 2003, 14, 980-991.], to enable its characterization.

The general principles behind the use of the fixed-charge peptide ion derivatization approach for directed CID MS/MS fragmentation are demonstrated here for the selective identification and differential quantitation of side chain fixed-charge sulfoniiun ion derivatives of peptides containing methionine (fixed-charge sulfonium ion derivatives of the amino acids methionine and cysteine, which were observed to fragment exclusively via neutral loss of the side chain CH₃SR, where R was a substituted alkyl group [Reid, G. E., Simpson, R. J. and O'Hair, R. A. J. J. Am. Soc. Mass Spectrom. 2000, 11, 1047-1060.; O'Hair R. A. J. and Reid, G. E. Eur. Mass Spectrom. 1999, 5, 325-334.], have previously been employed for the independent gas-phase synthesis of potential product ions involved in the side chain fragmentation reactions of amino acids.). Comparable strategies for the selective analysis of peptides containing other amino acids, those that contain post translationally modified amino acids, as well as for the characterization of cross-linked peptides are also described.

The present invention provides methods of analysis of amino acids, peptides or proteins, the methods comprising:
(1) derivatizing a mixture of amino acids, peptides or proteins, to form at least one amino acid, peptide or protein derivatized to contain a fixed-charge ion, other than at the C-terminal or N-terminal end thereof;
(2) introducing the mixture of amino acids, peptides or proteins containing at least one amino acid, peptide or protein derivatized to contain a fixed-charge ion other than at the C-terminal or N-terminal end thereof, to a mass spectrometer;
(3) passing the mixture of amino acids, peptides or proteins containing at least one amino acid, peptide or protein derivatized to contain a fixed-charge ion, other than at the C-terminal or N-terminal end thereof, through a first mass resolving spectrometer to select precursor protein or peptide ions having a first desired mass-to-charge ratio;
(4) subjecting the precursor ions of the first mass to charge ratio to dissociation to form a product ion having a characteristic second mass-to-charge ratio that is characteristic of a fragmentation occurring at a site adjacent to the fixed charge; and
(5) detecting the product ions having the second mass-to-charge ratio.

The product ion having the second characteristic mass-to-charge ratio may be either a charged amino acid, peptide or protein containing product ion formed by neutral loss of the fixed charge from the precursor ion, or a product ion formed by charged loss of the fixed charge from the precursor ion.

Preferably, the method of the invention comprises the further step of:
(6) determining the identity of the peptide or protein.

Step (6) may be performed by first repeating steps (1), (2), (3) and (4) and then subjecting the product ion having the second characteristic mass-to-charge ratio formed by (i) neutral loss from the precursor, which will have a charge state the same as that of the precursor, or (ii) the complementary product ion to the charged product ion formed by charged loss from the precursor ion, which corresponds to a protein or peptide containing product ion having a charge state one lower than the precursor, to a further stage of dissociation to form a series of product ions having a range of mass to charge ratios, for the purpose of determining the amino acid sequence of the peptide and subsequently, the identity of its protein of origin.

Alternatively, step (6) may be carried out by use of high resolution mass analyzers to obtain mass accuracies of approximately 1-5 ppm on the product ion detected in step (5), or its complementary product ion (i.e., to derive an “accurate product ion mass tag”). This, coupled with database searching, may be employed for subsequent identification of those peptides found to contain a fixed charge derivative. Previously, in cases where an “accurate mass tag” has been obtained for a precursor ion, and the presence of a particular amino acid is known (for example the presence of a cysteine residue), the specificity of database searching algorithms can be improved such that unambiguous identification of the protein from which the peptide is derived has been achieved from this information alone.

The amino acid, peptide or protein ion may be dissociated by any suitable dissociation method including, but not limited to, collisions with an inert gas (known as collision-induced dissociation (CID or collisionally-activated dissociation (CAD); (ii) collisions with a surface (known as surface-induced dissociation or SID); (iii) interaction with photons (e.g. via a laser) resulting in photodissociation; (iv) thermal/black body infrared radiative dissociation (BIRD), and (v) interaction with an electron beam, resulting in electron-induced dissociation for singly charged cations (EID), electron-capture dissociation (ECD) for multiply charged cations, or combinations thereof.

The methods of analysis of the present invention may be used for amino acid, peptide or protein identification, differential quantitation, analysis of post translational modification status, analysis of cross-linking status or interaction of proteins.

The present invention provides methods of analysis of amino acids, peptides or proteins containing a fixed charge derivative, at a site other than at the C-terminal or N-terminal end thereof.

The fixed-charge derivative may be contained on the side-chain of a selected amino acid, or a side-chain of a selected amino acid residue contained within a protein or peptide. Preferably the selected amino acid residue is that of a “rare” anmino acid, as described in more detail below. The fixed-charge derivative may be contained on a side-chain of a post translational modified amino acid residue, as described in more detail below. The fixed-charge derivative may be on a cross-link contained betwven two proteins or peptides, as described in more detail below.

The selected amino acid residue may be one containing a S atom in the side chain thereof. Preferred amino acid residues are methionine, cysteine, homocysteine or selenocysteine. The selected amino acid residue may also be tryptophan or tyrosine. The side chain may also contain an S-alkyl group. Preferred amino acid residues are methionine, S-alkyl cysteine, S-alkyl homocysteine, S-alkyl tryptophan or S-alkyl tyrosine. Derivatization of the side chain of a selected amino acid residue to introduce a fixed-charge may be achieved by strategies known in the art.

The fixed-charge may also be contained on an O-linked post-translationally modified amino acid residue (for example, a dehydroalanine residue formed by felieination from an O-linked post-translationally modified serine amino acid residue, or a dehydroamino-2-butyric acid residue formed by β-elimination from an O-linked post-translationally modified threonine amino acid residue) contained within a protein or peptide. Derivatization of a formerly post-translationally modified amino acid residue to introduce a fixed-charge may be achieved by strategies known in the art.

The fixed-harge may also be contained within a cross-linking reagent, or a cross-link contained between two amino acids, peptides or proteins. Derivatization of a cross-linking reagent or a cross-link contained between two amino acids, peptides or proteins to introduce a fixed-charge may be achieved by strategies known in the art.

Non-limiting examples of the fixed-charge include a sulfonium ion, a quaternary alkylammonium or a quaternary alkylphosphonium ion.

Analysis of the amino acid, peptide or protein ion may be performed by tandem mass spectrometry. The tandem mass spectrometer may be equipped with electrospray ionization (ESI) or matrix assisted laser desorption ionization (MALDI) interfaces to transfer the protein or peptide ion from solution into the gas-phase. Mass analysers that are applicable to tan.dem mass spectrometry fall into two basic categories: tandem-in-space and tandem-in-time. Combinations of these types also can be used. Tandem-in-space mass spectrometers have discreet mass analysers for each stage of mass spectrometry; examples include sector (commonly double focusing sector and “hybrid” combinations of sector and quadrupole analyser instruments), time of flight and triple quadrupole instruments, as well as “hybrid” combinations of time of flight and quadrupole instruments. Tandem-in-time mass instruments have only one mass analyser, and each stage of mass spectrometry takes place in the same region, but is separated in time via a sequence of events. Examples of tandem in time mass analysers include both two- and three-dimensional quadrupole ion trap and Fourier-transform ion cyclotron resonance (FT-ICR) mass spectrometers.

The methods of the invention in certain embodiments also include one or more steps of protein extraction, protein separation, reduction and alkylation of cysteine disulfides and/or digestion.

The fixed-charge derivatization approach in the method of the present invention may be applied to the quantitation of differential protein expression based on the incorporation of suitable isotopic (e.g., ¹³C, ¹⁵N, ²H) or structural labels to the fixed charge. The fixed-charge derivatization approach in the method of the present invention may be also applied to the identification and quantitation of post translational modification status in proteins by incorporation of the fixed-charge derivative, via the β-elimination/Michael addition method for forming mass spectrometry stable derivatives of O-phosphorylated and O-glycosylated serine, or O-phosphorylated and O-glycosylated threonine, for example, that is described in Meyer, H., Hoffinan-Posorske, E., Korte, H. and Heilmeyer, L. J. FEBS. Lett. 1986, 204, 61-66., the disclosure of which is incorporated herein by reference. The fixed-charge derivatization approach in the method of the present invention may also be applied to the identification and characterization of protein-protein interactions via incorporation of the fixed-charge derivative into a suitable cross-linking reagent.

As already mentioned above, tandem mass spectrometry of the fixed-charge derivatized peptides results in exclusive formation of a product ion upon dissociation that is characteristic of fragmentation at the fixed-charge site upon dissociation, thereby allowing specific identification of only those peptides containing the derivatization, without need for prior resolution or otherwise enrichment of the complex mixture prior to analysis.

The method of the present invention, in one particular embodiment, will be described below with reference to the selective identification and differential quantitation of peptides present at a range of different ratios and by the incorporation of isotopically labelled versions of the fixed-charge derivative, to yield a ‘light’ form (containing only natural isotopes), and a ‘heavy’ form (containing isotopic or structural labels incorporated into the substituent), with selective identification and differential quantitation of peptide levels performed by neutral loss mode tandem mass spectrometry. Application of the general approach to the analysis of post translational modification status as well as selective identification of protein-protein interactions by cross-linking analysis are also described.

In preferred embodiments of the invention, the foregoing methods are performed with derivatization of amino acids, peptides or proteins using a substituted acetophenone, or a salt thereof, or a solvate thereof, having the following formula: 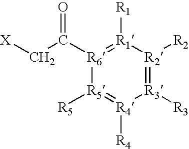
X preferably is any halogen, sulfonic ester, perchlorate ester or chlorosulfonate. In certain embodiments, R₁-R₅ are H, and R₁′-R₆′ are ¹²C. In other embodiments, the substituted acetophenone is an isotopically encoded substituted acetophenone, or a salt thereof, or a solvate thereof, preferably in which at least one of, more preferably two or more, and more preferably still at least three of R₁-R₅ are ²H, and R₁′-R₆′ are ¹²C, or in which R₁-R₅ are H, and at least one of, preferably two or more, and more preferably at least three of R₁′-R₆′ are ¹³C.

In certain embodiments, at least one of R₁-R₅ is a functional group containing an atom other than hydrogen or carbon.

Preferably the substituted acetophenone is water soluble. Certain water soluble molecules inchlde the foregoing molecules in which R₁ is SO₂H, and R₂-R₅ are H, and R₁′-R₆′ are ¹²C; R₁ is H, R₂ is SO₂H, and R₃-R₅ are H, and R₁′-R₆′ are ¹²C; R₁₋₂ are H, R₃ is SO₂H, and R₄-R₅ are H, and R₁′-R₆′ are ¹²C; R₁ is SO₃H, and R₂-R₅ are H, and R₁′-R₆′ are ¹²C; R₁ is H, R₂ is SO₃H, and R₃-R₅ are H, and R₁′-R₆′ are ¹²C; or R₁₋₂ are H, R₃ is SO₃H, and R₄-R₅ are H, and R₁′-R₆′ are ¹²C.

Isotopically encoded water soluble substituted acetophenone molecules include the foregoing wherein R₁ is SO₂H, and at least one of, and preferably at least three of R₂-R₅ are ²H, and R₁′-R₆′ are ¹²C; R₁ is SO₂H, and R₂-R₅ are H, and at least one of, and preferably at least three of R₁′-R₆′ are ¹³C; R₂ is SO₂H, and at least one of, and preferably at least three of R₁ and R₃-R₅ are ²H, and R₁′-R₆′ are ¹²C; R₂ is SO₂H, and R₁ and R₃-R₅ are H, and at least one of, and preferably at least three of R₁′-R₆′ are ¹³C; R₃ is SO₂H, and at least one of, and preferably at least three of R₁-R₂ and R₄-R₅ are ²H, and R₁′-R₆′ are ¹²C; R₃ is SO₂H, and R₁-R₂ and R₄-R₅ are H, and at least one of, and preferably at least three of R₁′-R₆′ are ¹³C; R₁ is SO₃H, and at least one of, and preferably at least three of R₂-R₅ are ²H, and R₁′-R₆′ are ¹²C; R₁ is SO₃H, and R₂-R₅ are H, and at least one of, and preferably at least three of R₁′-R₆′ are ¹³C; R₂ is SO₃H, and at least one of, and preferably at least three of R₁ and R₃-R₅ are ²H, and R₁′-R₆′ are ¹²C; R₂ is SO₃H, and R₁ and R₃-R₅ are H, and at least one of, and preferably at least three of R₁′-R₆′ are ¹³C; R₃ is SO₃H, and at least one of, and preferably at least three of R₁-R₂ and R₄-R₅ are ²H, and R₁′-R₆′ are ¹²C; or R₃ is SO₃H, and R₁-R₂ and R₄-R₅ are H, and at least one of, and preferably at least three of R₁′-are ¹³C.

In these molecules, it will be understood that other combinations and amounts of the listed substituents are possible and the invention embraces such combinations.

According to another aspect of the invention, substituted acetophenones (or salts thereof, or solvates thereof) are provided that have the following formula: 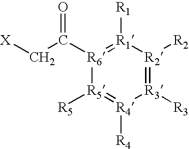
wherein X is a sulfonic ester, perchlorate ester or chlorosulfonate. Preferred substituted acetophenones are those in which R₁-R₅ are H, and R₁′-R₆′ are ¹²C.

The invention also includes substituted acetophenones that are isotopically encoded substituted acetophenones, salts thereof, and solvates thereof. Certain preferred substituted isotopically encoded acetophenones are those in which at least one of, and preferably at least three of R₁-R₅ are 2H, and R₁′-R₆′ are ¹²C, or those in which R₁-R₅ are H, and at least one of, and preferably at least three of R₁′-R₆′ are ¹³C.

The invention further includes water soluble substituted acetophenones, salts thereof, and solvates thereof, having the following formula:

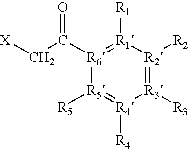

In some embodiments, X is any halogen, sulfonic ester, perchlorate ester or chlorosulfonate. In preferred embodiments, X is Br or I.

In other preferred embodiments, the water soluble substituted acetophenones are those in which R₁ is SO₂H, R₂-R₅ are H, and R₁′-R₆′ are ¹²C; R₁ is H, R₂ is SO₂H, R₃-R₅ are H, and R₁′-R₆′ are ¹²C; R₁₋₂ are H, R₃ is SO₂H, R₄-R₅ are H, and R₁′-R₆′ are ¹²C; R₁ is SO₃H, R₂-R₅ are H, and R₁′-R₆′ are ¹²C; R₁ is H, R₂ is SO₃H, R₃-R₅ are H, and R₁′-R₆′ are ¹²C; or R₁₋₂ are H, R₃ is SO₃H, R₄-R₅ are H, and R₁′-R₆′ are ¹²C.

The water soluble substituted acetophenone also can be isotopically encoded forms of those described herein, preferably wherein X is any halogen, sulfonic ester, perchlorate ester or chlorosulfonate. In particular, the isotopically encoded water soluble substituted acetophenones are those in which R₁ is SO₂H, at least one of, and preferably at least three of R₂-R₅ are ²H, and R₁′-R₆′ are ¹²C; R₁ is SO₂H, R₂-R₅ are H, and at least one of, and preferably at least three of R₁′-R₆′ are ¹³C; R₂ is SO₂H, at least one of, and preferably at least three of R₁ and R₃-R₅ are ²H, and R₁′-R₆′ are ¹²C; R₂ is SO₂H, R₁ and R₃-R₅ are H, and a least one of, and preferably at least three of R₁′-R₆′ are ¹³C; R₃ is SO₂H, at least one of, and preferably at least three of R₁-R₂ and R₄-R₅ are ²H, and R₁′-R₆′ are ¹²C; R₃ is SO₂H, R₁-R₂ and R₄-R₅ are H, and at least one of, and preferably at least three of R₁′-R₆′ are ¹³C; R₁ is SO₃H, at least one of, and preferably at least three of R₂-R₅ are ²H, and R₁′-R₆′ are ¹²C; R₁ is SO₃H, R₂-R₅ are H, and at least one of, and preferably at least three of R₁′-R₆′ are ¹³C; R₂ is SO₃H, at least one of, and preferably at least three of R₁ and R₃-R₅ are ²H, and R₁′-R₆′ are ¹²C; R₂ is SO₃H, R₁ and R₃-R₅ are H, and at least one of, and preferably at least three of R₁′-R₆′ are ¹³C; R₃ is SO₃H, at least one of, and preferably at least three of R₁-R₂ and R₄-R₅ are ²H, and R₁′-R₆′ are ¹²C; R₃ is SO₃H, R₁-R₂ and R₄-R₅ are H, and at least one of, and preferably at least three of R₁′-R₆′ are ¹³C.

In these molecules, it will be understood that other combinations and amounts of the listed substituents are possible and the invention embraces such combinations.

The invention in another aspect also includes reagent kits for analysis of amino acids, peptides or proteins by mass spectrometry. The kits include one or more containers containing the substituted acetophenones described herein.

Certain reagent kits for analysis of amino acids, peptides or proteins by mass spectrometry of the invention include a container containing a substituted acetophenone, or a salt thereof, or a solvate thereof, having the following formula: 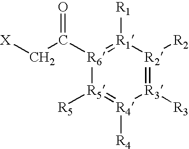
wherein X is a halide, preferably Br or I. Certain preferred substituted acetophenones in the kits are those in which R₁-R₅ are H, and R₁′-R₆′ are ¹²C. Other preferred substituted acetophenones in the kits are isotopically encoded substituted acetophenones, or salts thereof, or solvates thereof, preferably those in which at least one of, and preferably at least three of R₁-R₅ are ²H, and R₁′-R₆′ are ¹²C, or in which R₁-R₅ are H, and at least one of, and preferably at least three of R₁′-R₆′ are ¹³C. The water soluble derivatives of the substituted acetophenones listed above also can be included in the kits. Instructions for use of the substituted acetophenones in the analysis of amino acids, peptides or proteins by mass spectrometry also may be included.

The reagent kit fturther can contain one or more containers containing: cysteine disulfide reducing agents, cysteine alkylating reagents, proteases or chemical cleavage agents, and/or solvents. Tne cysteine disulfide reducing agents preferably include dithiothreitol (DTT), β-mercaptoethanol, tris-carboxyethyl phosphine (TCEP), and/or tributylphosphine (TBP). The cysteine alkylating reagents preferably include alkylhalides (e.g. iodoacetic acid, iodoacetamide), vinylpyridine and/or acrylamide. The proteases or chemical cleavage agents preferably include trypsin, Endoproteinase Lys-C, Endoproteinase Asp-N, Endoproteinase Glu-C, pepsin, papain, thermolysin, cyanogen bromide, hydroxylamine hydrochloride, 2-[2′-nitrophenylsulfenyl]-3-methyl-3′-bromoindole (BNPS-skatole), iodosobenzoic acid, pentafluoropropionic acid and/or dilute hydrochloric acid. The solvents preferably include urea, guanidine hydrochloride, acetonitrile, methanol and/or water.

The invention also includes amino acids, or peptides comprising an amino acid, derivatized to include a side chain fixed-charge sulfonium ion, quatemary alkylammonium ion or quaternary alkylphosphonium ion. Preferably the amino acid is derivatized using the substituted acetophenone described herein. In particularly preferred embodiments, the amino acid is derivatized using a substituted acetophenone, or a salt thereof, or a solvate thereof, having the following formula: 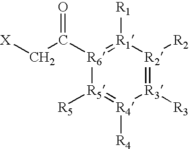
wherein X is a halide, preferably Br or I. Certain preferred substituted acetophenones used to derivatize the amino acids or peptides are those in which R₁-R₅ are H, and R₁′-R₆′ are ¹²C. Other preferred substituted acetophenones used to derivatize the amino acids or peptides are isotopically encoded substituted acetophenones, or salts thereof, or solvates thereof, preferably those in which at least one of, and preferably at least three of R₁-R₅ are ²H, and R₁′-R₆′ are ¹²C, or in which R₁-R₅ are H, and at least one of, and preferably at least three of R₁′-R₆′ are ¹³C. The water soluble derivatives of the substituted acetophenones listed above also can be used to derivatize the amino acids or peptides. In preferred embodiments, the amino acid derivative is isotopically encoded.

The invention in another aspect provides methods for providing an internal standard in a mass spectrometer method comprising adding to a sample a predetermined quantity of the fixed charge derivatized amino acid or peptide described above.

The present invention will now be described with reference to particular embodiments, however, the method of the present invention is not limited to these particular embodiments.

### BRIEF DESCRIPTION OF THE DRAWINGS

FIG. 1. Quadrupole ion trap tandem mass spectrometry of singly charged GAILMGAILA (SEQ ID NO:1) and its methionine side chain fixed-charge derivative. (A) CID MS/MS of the [M+H]⁺ ion. (13) CD MS/MS of the methionine side chain fixed-charge acetophenone (AP) sulfonium [M(AP)]⁺ ion. (inset) CID MS³ of the [M−CH₃S(AP)]⁺ product ion in FIG. 1B.

FIG. 2. Quadrupole ion trap tandem mass spectrometry of doubly charged GAILMGAILA (SEQ ID NO:1) and its methionine side chain fixed-charge derivative. (A) CID MS/MS of the [M+2H]²⁺ ion. (B) CID MS/MS of the methionine side chain fixed-charge acetophenone (AP) sulfonium [M(AP)+H]²⁺ ion. (inset) CID MS³ of the [M−CH₃S(AP)]²⁺ product ion in FIG. 2B.

FIG. 3. Quadrupole ion trap tandem mass spectrometry of singly charged GAILMGAILK (SEQ ID NO:2) and its methionine side chain fixed-charge derivative. (A) CID MS/MS of the [M+H]⁺ ion. (13) CID MS/MS of the methionine side chain fixed-charge acetophenone (AP) sulfonium [M(AP)]⁺ ion. (inset) CID MS³ of the [M−CH₃S(AP)]⁺ product ion in FIG. 3B.

FIG. 4. Quadrupole ion trap tandem mass spectrometry of doubly charged GAILMGAILK (SEQ ID NO:2) and its methionine side chain fixed-charge derivative. (A) CID MS/MS of the [M+2H]²⁺ ion. (B) CID MS/MS of the mef.hionine side chain fixed-charge acetophenone (AP) sulfonium [M(AP)+H]²⁺ ion. (inset) CID MS³ of the [M−CH₃S(AP)]²⁺ product ion in FIG. 4B.

FIG. 5. Quadrupole ion trap tandem mass spectrometry of the triply charged acetophenone (AP) sulfonium ion derivative of GAILMGAILK (SEQ ID NO:2). (A) CID MS/MS of the [M(AP)+2H]³⁺ ion. (inset) CID MS³ of the [M−CH₃S(AP)]³⁺ product ion.

FIG. 6. Quadrupole ion trap tandem mass spectrometry of singly charged GAILMGAILR (SEQ ID NO:3) and its methionine side chain fixed-charge derivative. (A) CID MS/MS of the [M+H]⁺ ion. (B) CID MS/MS of the methionine side chain fixed-charge acetophenone (AP) sulfonium [M(AP)]⁺ ion. (inset) CID MS³ of the [M−CH₃S(AP)]⁺ product ion in FIG. 6B.

FIG. 7. Quadrupole ion trap tandem mass spectrometry of doubly charged GAILMGAILR (SEQ ID NO:3) and its methionine side chain fixed-charge derivative. (A) CID MSIMS of the [M+2H]²⁺ ion. (B) CID MS/MS of the methionine side chain fixed-charge acetophenone (AP) sulfonium [M(AP)+H]²⁺ ion. (inset) CID MS³ of the [M−CH₃S(AP)]²⁺ product ion in FIG. 7B.

FIG. 8. Quadrupole ion trap tandem mass spectrometry of the triply charged acetophenone (AP) sulfonium ion derivative of GAILMGAILR (SEQ ID NO:3). (A) CID MS/MNS of the [M(AP)+2H]³⁺ ion. (inset) CHD MS³ of the [M−CH₃S(AP)]³⁺ product ion.

FIG. 9. (A) Energy resolved CID MS/MS of the doubly charged methionine side chain fixed-charge acetophenone (AP) sulfonium [M(AP)+H]²⁺ ion of GAILMGAILK (SEQ ID NO:2). (B) CD MS/MS product ion spectra of the [M(AP)+H]²⁺ ion obtained at a collision energy (laboratory frame) of 12V. (C) CID MS/MS product ion spectra of the [M(AP)+H]²⁺ ion obtained at a collision energy (laboratory frame) of 22V.

FIG. 10. Energy resolved CID MS/MS of the doubly charged [M+2H]²⁺ ions of GAILAGAILK (SEQ ID NO:5) and GAILMGAILK (SEQ ID NO:2).

FIG. 11. (A) Total ion current trace from a 200 μm I.D. capillary RP-HPLC·MS analysis of 10 pmol of a reduced and S-carboxyamidomethylated tryptic digest of bovine serun albumin, following derivatization using bromoacetophenone. Individual mass spectra for two regions of the total ion current chromatogram, indicated by arrows, are shown in FIGS. 11B and 11C.

FIG. 12. (A) Total ion current trace from a 200 μm I.D. capillary RP-HPLC—neutral loss scan mode CID MS/MS analysis of 10 pmol of a reduced and S-carboxyamidomethylated tryptic digest of bovine serum albumin, following derivatiiation using bromoacetophenone. The peptides are IETMR (SEQ ID NO:9), ETYGDMADCCEK (SEQ ID NO:10), TVMENFVAFVDK (SEQ ID NO:11), and MPCTEDYLSLILNR (SEQ ID NO:12). Individual neutral loss scan mode mass spectra for two regions of the total ion current chromatogram, indicated by arrows, are shown in FIGS. 12B and 12C.

FIG. 13. (A) Mass spectrum and (B) neutral loss scan mode CID MS/MS spectra (neutral loss of 83.0 Da and 85.5 Da for the doubly charged d₀- and d₅-containing [M(AP)+H]²⁺ ions, and neutral loss of 55.3 Da and 57.0 Da for the triply charged d₀- and d₅-containing [M(AP)+2H]³⁺ ions) of a 1.0:1.0 pmol/μL mixture of d₀ and d₅ containing methionine side chain fixed-charge acetophenone (AP) sulfonium ion derivative of GAILMGAILR (SEQ ID NO:3).

FIG. 14. (A) Mass spectrum of a 1.0:0.1 pmol/μL mixture of d₀ and d₅ containing methionine side chain fixed-charge acetophenone sulfonium ion derivative of GAILMGAILR (SEQ ID NO:3). Expanded regions of the m/z range for the triply and doubly charged precursors are shown in panels (B) and (C) respectively.

FIG. 15. (A) Summed neutral loss scan mode CID MS/MS spectrum of a 1.0:0.1 pmol/μL mixture of d₀ and d₅ containing methionine side chain fixed-charge acetophenone sulfonium ion derivative of GAILMGAILR (SEQ ID NO:3). Individual neutral loss mass spectra for the triply and doubly charged precursors are shown in panels (B) and (C), respectively.

FIG. 16. (A) Total ion current trace from a 75 μm I.D. capillary RP-HPLC·MS analysis of a 1.0:1.0 pmol mixture of d₀ and d₅ containing methionine side chain fixed-charge acetophenone (AP) sulfonium ion derivative of GAILMGAILR (SEQ ID NO:3). (B) Summed mass spectra from the region indicated in panel (A). (C) Total ion current trace from a 75 μm I.D. capillary RP-HPLC—neutral loss scan mode CID MS/MS analysis of a 1.0:1.0 pmol mixture of d₀ and d₅ containing methionine side chain fixed-charge acetophenone (AP) sulfonium ion derivative of GAILMGAILR (SEQ ID NO:3). (D) Summed mass spectra from the region indicated in panel (C).

### EMBODIMENTS OF THE INVENTION

Definitions

Unless the context indicates otherwise, all technical and scientific terms used herein generally have the same meaning as commonly understood in the art to which the present invention belongs.

“Fixed-charge”, as used herein, includes any charge localised to a specific heteroatom contained within the protein or peptide, or to a specific heteroatom contained within the derivatization reagent (e.g., in solution or in the gas-phase), by the attachment of any moiety.

“Fixed charge derivatization”, as used here, means the introduction of a fixed charge as defined above. For example, the fixed charge may be introduced either by introducing a neutral reagent to subsequently form the fixed charge at a specific site within the protein or peptide, or by introduction of a reagent containing the fixed charge to a specific site within the protein or peptide.

The fixed charge derivative thus formed preferably has a structure such that it allows the exclusive formation of a product ion upon dissociation that is characteristic of the fixed charge derivative.

“Protein”, as used herein, means any protein, including, but not limited to peptides, enzymes, glycoproteins, hormones, receptors, antigens, antibodies, growth factors, etc., without limitation. Proteins may be endogenous, or produced from other proteins by chemical or proteolytic cleavage. Preferred proteins include those comprised of at least 15-20 amino acid residues. The term includes cross-linked proteins.

“Peptide” as used herein includes any substance comprising two or more amino acids atnd includes di-, tri-, oligo and polypeptides etc according to the number of amino acids linked by amide(s) bonds. Peptides may be endogenous, or produced from other peptides or proteins by chemical or proteolytic cleavage. Preferred peptides include those comprised of up to 15-20 amino acid residues. The term includes cross-linked peptides.

When the amino acids are α-amino acids, either the L-optical isomer or the D-optical isomer can be used. The L-isomers are generally preferred. For a general review, see, [Spatola, A. F., in Chemistry and Biochemistry of amino acids, peptides and proteins. 1983, B. Weinstein, eds., Marcel Dekker, New York, p. 267.]

The term “alkyl” is used herein to refer to a branched or unbranched, saturated or unsaturated, monovalent hydrocarbon. Suitable alkyl groups include, for example, structures containing one or more methylene, methine and/or methyne groups. Branched structures have a branching motif similar to i-propyl, t-butyl, i-butyl, 2-ethylpropyl, etc. As used herein, the term encompasses “substituted alkyls,” and “cyclic alkyl.”

“Substituted alkyl” refers to alkyl as just described including one or more substituents such as, for example, alkyl, aryl, acyl, halogen (i.e., alkylhalos, e.g., CF₃), hydroxy, amino, amide, alkoxy, alkylamino, acylamino, thioamido, acyloxy, aryloxy, aryloxyalkyl, ether, ester, disulfide, mercapto, thia, aza, oxo, both saturated and unsaturated cyclic hydrocarbons, heterocycles and the like. These groups may be attached to any carbon or substituent of the alkyl moiety. Additionally, these groups may be pendent from, or integral to, the alkyl chain. For the purpose of covalent solid phase pre-emichment of fixed charge derivatives, the substituted alkyl group may be covalently attached to an insoluble bead or polymer, and contain a chemical or photochemical cleavage site between the insoluble bead or polymer and the alkyl group.

The term “aryl” is used herein to refer to an aromatic substituent, which may be a single aromatic ring or multiple aromatic rings which are fused together, linked covalently, or linked to a common group such as a methylene or ethylene moiety. The common linking group may also be a carbonyl as in acetophenone. The aromatic ring(s) may include phenyl, naphthyl, biphenyl, diphenylmethyl and benzophenone among others. The term “aryl” encompasses “arylalkyl” and “substituted aryl.”

“Substituted aryl” refers to aryl as just described including one or more functional groups such as lower alkyl, acyl, halogen, alkylhalos (e.g. CF₃), hydroxy, amino, alkoxy, alkylamino, acylamino, acyloxy, phenoxy, mercapto and both saturated and unsaturated cyclic hydrocarbons which are fused to the aromatic ring(s), linked covalently or linked to a common group such as a methylene or ethylene moiety The linking group may also be a carbonyl such as in cyclohexyl phenyl ketone. The term “substituted aryl” encompasses “substituted arylalkyl.”

The term “arylalkyl” is used herein to refer to a subset of “aryl” in which the aryl group is attached to another group by an alkyl group as defined herein.

“Substituted arylalkyl” defines a subset of “substituted aryl” wherein the substituted aryl group is attached to another group by an alkyl group as defined herein.

The term “acyl” is used to describe a ketone substituent, —(O)R, where R is alkyl or substituted alkyl, aryl or substituted aryl as defined herein.

Abbreviations

CID Collision Induced Dissociation
ESI Electrospray Ionization
MALDI Matrix Assisted Laser Desorption Ionization
MS Mass spectrometry
MS/MS Tandem mass spectrometry
MSⁿ Multistage tandem mass spectrometry, where n>2
   Materials and Methods.
   Materials

The model synthetic peptides (GAILMGAILA (SEQ ID NO: 1), GAILMGAILK (SEQ ID NO:2), GAILMGAILR (SEQ ID NO:3), GAILAGAILA (SEQ ID NO:4), GAILAGAILK (SEQ ID NO:5) and GAILAGAILR (SEQ ID NO:6)) were obtained from Auspep (Melbourne, Vic, Australia) and used directly without further purification. Iodomethane, iodoethane, iodobenzene, iodomethylbenzene, d₅-acetophenone and bromoacetophenone were purchased from Aldrich (Castle Hill, NSW, Australia). Polymer supported pyridyl bromide perbromide, iodoacetic acid and iodoacetamide were from Sigma (St. Louis, Mo., USA). Methanol, and acetonitrile (Chromar grade) were purchased from Mallinkrodt (Paris, Ky., USA). Formic acid and acetic acid were obtained from BDH Laboratories (Poole, England). All solutions were prepared using deionised water purified by a tandem Milli-Q and Milli-RO system (Millipore, Bedford, Mass., USA).

Synthesis of d₅-bromoacetophenone.

d₅-bromoacetophenone was synthesized according to the method of Frechet et al [Frechet, J. M. J., Farrall, M. J. and Nuyens, L. J. J. Macromol. Sci.-Chem. 1977, A11, 507-514.] Briefly, 1.25 mL of d₅-acetophenone was added to 4.8 g Polymer supported pyridyl bromide perbromide (˜3 meq Br₃⁻/g resin) in 30 mL methanol then allowed to react with stirring at room temperature for 4 hours. The resultant α-bromo ketone was obtained in pure form by filtration of the reaction mixture and evaporation of the solvent, followed by recrystallization prior to use.

Side Chain Fixed-charge Derivatization of Methionine Containing Peptides.

Side chain fixed-charge sulfonium ion derivatives of methionine-containing peptides were produced by the addition of 10 μL of a 1M solution of alkylating reagent to 100 μg of is peptide dissolved in 100 μL of aqueous 20% acetic acid containing 30% CH₃CN. The reaction was allowed to proceed for 16 hours at room temperature after which the sample was diluted and then introduced to the mass spectrometer with no further purification.

Mass Spectroinetry

Mass spectrometric analysis was performed using either a (i) quadrupole ion trap (Finnigan-MAT model LCQ-DECA, San Jose, Calif.), (ii) quadrupole-time-of-flight (icromass model Q-TOF2, Manchester, UK), or (ii) triple quadrupole (Finnigan model TSQ, San Jose, Calif.) mass spectrometer, all equipped with electrospray ionization interfaces.

Quadrupole Ion Trap Mass Spectrometry.

Samples (10 pmol/μL in 50:50:1 H₂O:CH₃CN:acetic acid) were introduced to the mass spectrometer at 2 μL/min. The ESI conditions were optinmized to maximize the intensity of the ion of interest. Typical conditions were: spray voltage −5 kV, Nitrogen sheath gas, 30 psi, heated capillary temperature 150° C. MS/MS and MS³ experiments were performed on mass selected ions using standard isolation and excitation procedures. All spectra collected were the average of 10 scans.

Quadrupole-time-of-flight Mass Spectrometry.

Spectra were acquired using the V-optics mode of the time-of-flight mass analyser following infusion of samples (same concentration as solution composition as for ion trap experiments) at 1 μL/min. Electrospray interface conditions were optimized to maximize the intensity of the ion of interest. Typical conditions were: spray voltage, −4.5 kV; nitrogen source gas, 1 psi; cone gas, 100 (arbitrary units); source temperature, 50° C.; desolvation temperature, 150° C.; cone voltage, 50V (singly charged ions), 30V (doubly charged ions) and 20V (triply charged ions). Energy resolved CID MS/MS experiments were automatically acquired on an isolated isotopic envelope precursor ion population to allow determination of product ion charge states, using argon as the inert collision gas at a pressure of 10 psi. The collision energy was ramped from 4 to 58 V in 1V increments for singly charged ions, and from 4 to 20 V in 1V increments and from 20 to 46V in 2V increments for doubly and triply charged ions. Spectra obtained for each collision energy value are the average of 10 scans.

Triple Quadrupole Mass Spectrometry.

Samples (1 pmol/μL in 50:50:1 H₂O:CH₃CN:acetic acid) were introduced to the mass spectrometer by a home built nano-electrospray ionization source at a flow rate of 200 nL/min. The spray voltage was maintained at −1.8 kV. The heated capillary temperature was 150° C. The argon collision gas pressure was maintained at 1.5 mtorr. The instrument was operated under unit resolution conditions. Neutral loss mode MS/MS scans (neutral losses of 83 and 85.5 Da for d₀- and d₅-containing doubly charged ions, and 55.3 and 57 Da for d₀- and d₅-containing triply charged ions) were performed at collision energies of 18V and 13V, respectively. Product ion CID MS/MS spectra of peptide ions selectively identified by neutral loss scans were then acquired at 18V and 31V, and 13V and 18V for doubly and triply charged ions, respectively. All spectra shown are the average of 20 scans.

Capillary RP-HPLC was performed using (i) a colunui (200 μm I.D.×150 mm O.D fused silica), packed with Brownlee RP-300, 7 μm dimethyloctyl silica, developed at a flow rate of 3.6 μL min⁻¹ using a linear 60 minute gradient from 0-100% B, where solvent A was 0.1M aqueous acetic acid, and solvent B was 0.1M aqueous acetic acid containing 60% acetonitrile, or (ii) a column (75 μm I.D.×150 mm O.D fused silica), packed with Vydac C4, developed at a flow rate of 200 nL min⁻¹ using a linear 60 minute gradient from 0-100% B, where solvent A was 0.1M aqueous acetic acid, and solvent B was 0.1M aqueous acetic acid containing 60% acetonitrile.

Results and Discussion

Identification of Proteins by CID MS/MS of Their Fixed-charge Derivative Containing Peptides.

Side chain fixed-charge derivatization of peptides containing methionine has been employed here to demonstrate the general strategy for selective identification and quantitation of peptides by selective CID MS/MS dissociation. The rationale for using fixed-charge derivatives of methionine containing peptides is based on the idea that approaches for selective identification and quantitation of differential protein expression should be specific for the detection of peptides containing amino acids that are rare, thereby limiting the number of peptides required to be analyzed, yet providing comprehensive coverage of proteins in the sequence databases. Release 40.0 of the SWISS-PROT database contains a total of 101,602 protein entries. The occurrence of methionine in this database is 2.37%. In contrast, the occurrence of cysteine, the amino acid targeted in the popular ICAT and related methods for selective mass spectrometric detection and quantitation, is 1.63%. Of the individual database entries, 85.33% contain cysteine while 96.9% contain methionine. 98.35% coverage could be attained if proteins containing both amino acids were targeted for analysis, as only 1.65% of entries do not contain either methionine or cysteine. Therefore, approaches directed toward the identification of proteins containing methionine (or both methionine and cysteine) should cover a significantly greater fraction of the proteins represented in the database than approaches targeting for cysteine alone.

The main strategy devised here for the selective identification and subsequent differential quantitation of methionine-containing peptides, as well as for S-alkyl cysteine, by fixed-charge sulfonium ion derivatization (i.e., derivatives in which the side chain of methionine or S-alkyl cysteine has been alkylated) and tandem mass spectrometry, are outlined below. Comparable strategies for the selective identification and differential quantitation of cysteine containing peptides, as well as a slrategy for the selective identification and differential quantitation of tryptophan (1.21% occurrence) containing peptides, via sulfonium or quatemary alkyl fixed charge derivatization are also discussed.

Fixed-charge Sulfonium Ion Derivatization of Peptides Containing Methionine.

Identification of methionine-containing peptides via CID of its sulfonium ion fixed-charge derivative is attractive for several reasons. Methionine is a rare arnino acid (see above). The chemistry and biological applications of sulfonium ions are well documented, giving precedent for developing reagents to form these ions in applications involving specific io derivatization of peptides [Stirling, M. J. and Patai, S. The Chemistry of the Sulfoniurn Group, Wiley: New York, 1981.; Lundblad. Techniques in Protein Modification, Chapter 8 “The Modification of Methionine” CRC Press. Florida, 1995.; Liu T.-H. “The role of Sulfur in Proteins” In: The Proteins. Volume III, Neurath, Hill and Boeder. Eds., Academic NY, 1977, Chapter 3, pp 239-402.; Gundlach, H. G. Moore, S, and Stein, W. H. J. Biol. Chem., 1959, 234, 1761-1764.; Lawson, W. B. Gross, E., Foltz, C. M. and Witkop, B. J. Am. Chem. Soc., 1961, 83, 1509-1510.; Lawson, W. B. Gross, E., Foltz, C. M. and Witkop, B. J. Am. Chem. Soc., 1962, 84, 1715-1718.; Gross E. Methods Enzymology 1967, 11, 238-255.; Degen, J. and Kyte, J. Anal. Biochem. 1978, 89, 529-539.; Kyte, J., Degen, J. and Harkins, R. N. Methods Enzymology 1983, 91, 367-377.]. The alkylation of methionine residues in proteins and peptides is specific at low pH regardless of the alkylating reagent used [Lundblad. Techniques in Protein Modification, Chapter 8 “The Modification of Methionine” CRC Press. Florida, 1995.; Liu T.-H. “The role of Sulfur in Proteins” In: The Proteins. Volume III, Neurath, Hill and Boeder. Eds., Academic NY, 1977, Chapter 3, pp 239-402.]. The reason for this selectivity is that the rate of alkylation of the thioether sulfuir atom of the methionine side chain is virtually independent of pH (even down to pH1) whereas the reactivity of all other nucleophilic functional groups (e.g. cysteine, lysine and histidine residues) decreases at low pH (due to protonation). Previously, sulfonium ions have been exploited to: (a) tag methionine sites in peptides and proteins [Lundblad. Techniques in Protein Modification, Chapter 8 “The Modification of Methionine” CRC Press. Florida, 1995.; Liu T.-H. “The role of Sulfur in Proteins” In: The Proteins. Volume III, Neurath, Hill and Boeder. Eds., Academic NY, 1977, Chapter 3, pp 239-402.; Gundlach, H. G. Moore, S, and Stein, W. H. J. Biol. Chem., 1959, 234, 1761-1764.; Rogers, G. A. Shaltiel, N, and Boyer, P. D. J. Biol. Chem., 1976, 251, 5711-5717.; Toennies, G. and Kolb, J. J. J. Am. Chem. Soc. 1945, 67, 849-851.; Lavine, T. F. Floyd, N. F, and Cammaroti, M. S. J. Am. Chem. Soc., 1945, 67, 849-851.; Lawson, W. B. and Schramm, H. J. J. Am. Chem. Soc., 1962, 84, 2017-2018.]; (b) to induce cleavage of peptide bonds adjacent to methionine residues via the solution phase fragmentation of the sulfonium ions. [Lawson, W. B. Gross, E., Foltz, C. M. and Witkop, B. J. Am. Chem. Soc., 1961, 83, 1509-151 O.; Lawson, W. B. Gross, E., Foltz, C. M. and Witkop, B. J. Am. Chem. Soc., 1962, 84, 1715-1718.; Jendrek, J. P., Barker, R. H. and Altschul, A. M. Biochim Biophys Acta, 1967, 136, 409-411.; Gross E. Methods Enzymology 1967, 11, 238-255.] The most famous and widely studied system is the cyanogen bromide reaction [Gross E. Methods Enzymology 1967, 11, 238-255.], although other alkylating reagents have been used to similar effect [Tang, J. R. and Hartley, B. S. Biochem. J. 1967, 102, 593.; Tang, J. R and Hartley, B. S. Biochem. J. 1970, 118, 611.]. Note that related reactions have been observed in peptide syntheses [Gairi, M., Lloyd-Williams, P., Albericio, F. and Giralt, E. Tetrahedron Lett. 1994, 35, 175-178.], as an autolytic protein cleavage reaction [Taylor, K L.; Pohl, J; Kinkade, J. M., J. Biol. Chem. 1992, 267, 25282-25288.], in the purification of peptides [Tang, J. R and Hartley, B. S. Biochem. J. 1967, 102, 593.; Degen, J. and Kyte, J. Anal. Biochem. 1978, 89, 529-539.; Kyte, J., Degen, J. and Harkins, R. N. Methods Enzymology 1983, 91, 367-377.; Weinberger, S. R.; Viner, R. I. and Ho, P. Electrophoresis. 2002, 23, 3182-3192.], and to transform methionine residues to homocysteine residues [Chassaing, G; Lavielle, S; Marquet, A. J. Org. Chem. 1983, 48, 1757-60.]. Preliminary experiments involving sulfonium ions derivatives of methionine have been performed, using substituted alkyl halides R₁X (where X is the halogen and R is the substituted alkyl group) [O'Hair, R. A. J. and Reid, G. E. Eur. Mass Spectrom. 1999, 5, 325-334.]. The reagent used in these preliminary studies reacts specifically with the side chain of methionine in solution under acidic pH conditions to form a stable side chain sulfonium ion derivative (—CH(CH₂)₂S(CH₃)R₁⁺) (step (i) of Scheme 1). Under these conditions, the reagent does not react with the free thiol of cysteine containing peptides. The reagent may however, be used for derivatization of cysteine-containing peptides after initial S-alkylation of the cysteine thiol side chain under basic pH conditions to yield the derivative (—CHCH₂SR₂) (step (i) of Scheme 2). Further alkylation of these S-alkyl cysteine containing peptides under identical conditions to those employed for alkylation of methionine can then be used to form stable sulfonium ion derivatives of cysteine (—CHCH₂SR₁R₂⁺) [Foti, S., Saletti, R. and Marletta, D. Org. Mass Spectrom. 1991, 26, 903-907.; Lapko, V. N., Smith, D. L. and Smith, J. B. Mass Spectrom. 2000, 35, 572-575.], provided that the R₂ substituent used for the initial cysteine alkylation enhances sulfonium ion stability (i.e., the charge is stable) (step (ii) of Scheme 2). Importantly, conditions could be chosen so that the initial S-alkyl cysteine derivative is not stable to sulfonium ion formation, allowing specific derivatization of methionine containing peptides in cases where cysteines have been previously reduced and S-alkylated to enable efficient proteolysis during sample preparation. Neutral loss mode scan mode CID tandem mass spectrometry (MS/MS) scans, or post data acquisition neutral loss product ion detection software following conventional CID MS/MS, can be used to selectively identify methionine and cysteine containing peptides via the characteristic loss of CH₃SR₁ from methionine and R₁SR₂ from cysteine, with formation of [M+nH—CH₃SR]⁽ⁿ⁺¹⁾⁺ and [M+nH—R₁SR₂]⁽ⁿ⁺¹⁾⁺ product ions, respectively. Methionine and cysteine containing peptides can therefore be identified individually, through the use of an R₂ group that does not correspond to CH₃, (i.e., the side chain alkyl group of methionine) or simultaneously, via initial alkylation of cysteine where the R₂ group corresponds to —CH₃.

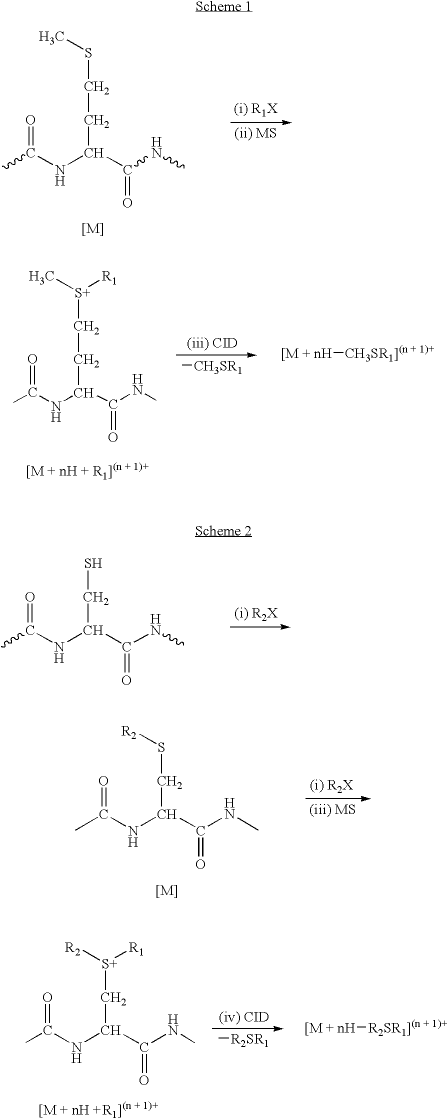

In order for this derivatization strategy to be successful, both the solution phase and gas phase chemistries of the resultant sulfonium ions must be considered. These solution and gas phase chemistries, as well as characterization of the fragmentation behaviour of the fixed-charge sulfonium ion derivatives of methionine containing peptides have been examined using a series of model peptides designed to resemble those resulting from tryptic digestion, i.e., the class of peptides most commonly employed for protein identification by tandem mass spectrometry. The amino acid sequences of the model peptides (GAILX₁GAILX₂ (SEQ ID NO:7)), where X₁ is either alanine or methionine, and X₂ is either alanine, lysine or arginine), were chosen to allow comprehensive examination of the effects of charge state and amino acid composition on the observed fragmentation behaviour.

Solution Phase Requirements for Sulfonium Ion Formation:

Reactions should be easy to perform using readily available reagents. Many of the potentially useful reagents are commercially available. Other potentially useful reagents may be synthesized by simple methods described in the literature. Importantly, the ability to incorporate a suitable isotopic label into the selected derivative is an important consideration in allowing subsequent quantitation of differential protein expression using the same approach. Thus, isotopically labelled derivatives of these reagents should be commercially available or readily synthesized. For example, isotopically labeled d₅-bromoacetophenone has been readily prepared here from d₅-acteophenone using the brominating reagent, poly(4-vinylpyridinium tribromide), in a one-step process (see Materials and Methods). Reactions should be fast, proceed to completion and the resultant sulfonium ions must be chemically and thermally stable in solution [Stirling, M. J. and Patai, S. The Chemistry of the Sulfonium Group, Wiley: New York, 1981.; Stirling C. J. M. Sulfonium Salts in Organic Chemistry of Sulfr Oae, S. Ed. Plenum Press NY 1977, Chapter 9, pp 473-525.; Capozzi, G, and Modena, G. Stud. Org. Chem. 1985, 19,246-298.]. Using the model peptide GAILMGAILK (SEQ ID NO:2), a range of potential alkylating reagents for methionine side chain fixed-charge sulfonium ion formation, iodomethane, iodoethane, iodobenzene, iodomethylbenzene, iodoacetic acid, iodoacetamide and bromoacetophenone have been evaluated. The peptide was reacted for 4, 8, 16, and 32 hours with a 100 fold molar excess of each alkylating reagent then analysed by mass spectrometry to determine the extent of reaction. After 16 hours, only the acetophenone sulfonium ion derivative had reacted to completion [March J. “Advanced Organic Chemistry”, 4th Ed., Wiley, New York, 1992, pp 343.; Halvorsen, S. J. Chem. Soc. Chem Comm, 1978, 327.; Yoh, L. Tetrahedron Lett, 1988, 29, 443 1 ]. The ethyl derivative was found to have the least reactivity and stability in solution (only 1% reaction after 16 hours). All other reagents ranged in reactivity between these two extremes. The acetophenone derived sulfonium ion of the peptide was found to be stable in solution for several weeks without significant degradation. As free SH groups of cysteine residues have been shown to effect the reversal of the alkylation of methionine residues [Scheitjter, A. and Aviram, I. FEBS Lett 1972, 21, 293-296.], and transfer of a methyl group from a sulfonium salt to a free sulihydryl acceptor is a well known possible side reaction [Toennies, G. and Kolb, J. J. J. Am. Chem. Soc., 1945, 67, 1141-1144.; Naider, F. and Bohak, Z. Biochemistry 1972, 11, 3208-3211.; Cantoni, G. L. Comp. Biochem. 1960, 1, 181.], the commonly employed step involving S-alkylation of cysteines prior to proteolysis may be a useful but necessary reaction.

Gas Phase Requiremenits for Sulfonium Ion Fragmentation

All sulfonium ions examined, including those with poor reactivity in solution, were found to be stable in the gas-phase so that they could be mass selected and manipulated for MS/MS experiments. All ions were found to give a unique fingerprint by fragmenting via the neutral loss of CH₃SR, (see Scheme 1). The loss of CH₃SR, from fixed-charge derivatives has been observed previously in small model systems [Reid, G. E., Simpson, R. J. and O'Hair, R. A. J. J. Am. Soc. Mass Spectrom. 2000, 11, 1047-1060.; O'Hair, R. A. J. and Reid, G. E. Eur. Mass Spectrom. 1999, 5, 325-334.], peptides [Foti, S., Saletti, R. and Marletta, D. Org. Mass Spectrom. 1991, 26, 903-907.] and proteins [Lapko, V. N., Smith, D. L. and Smith, J. B. Mass Spectrom. 2000, 35, 572-575.]. Note that these gas phase reaction have solution phase analogies as discussed above. Side reactions such as charge migration via intramolecular proton transfer, alkyl group transfer, as well as breaking of bonds to give stable alkyl cations or alkenes must be avoided [O'Hair, R. A. J., Freitas, M. A., Gronert, S., Schmidt, J. A. R. and Williams, T. D. J. Org. Chem., 1995, 60, 1990-1998.; Mudd, S. H. et. al. Biochem, 1966, 5, 1653.; Deakyne, C. A. et. al. J. Mol. Struct. 1999, 485/486, 33-41.; Markham, G. D. and Bock, C. W. J Phys Chem, 1993, 97, 5562-5569.; Katritzky, A. R. et. al. Int J Mass Spectrom, 1997, 165/166, 577-583.; Buckley, N. et. al. J. Org. Chem. 1996. 61, 2753-62.; Mestdagh, H. et. al. Org. Mass Spectrom. 1986, 21, 321-327.; Mestdagh, H. et. al. Org. Mass Spectrom. 1988, 23, 246-251.]. Proton transfer (Scheme 3, pathway 1) would give an sulfiur ylide. Although the acidity of the (CH₃)₃S⁺ ion (or the proton affinity of the conjugate base (CH₃)₂S(CH₂)) is unknown, sulfur ylides may be formed in solution by deprotonating sulfonium ions with bases such as hydroxide ion (HO—) or alkyl lithium reagents [Trost, B. M. and Melvin, L. S. “Sulfur Ylides: Emerging Synthetic Intermediates”. Academic Press: NY 1975.] Alkyl transfer (Scheme 3, pathway 2) is not likely to be a problem since intramolecular S_{N}2 rarely occur due to high energy transition states (co-linear geometry) [O'Hair, R. A. J., Freitas, M. A., Gronert, S., Schmidt, J. A. R. and Williams, T. D. J. Org. Chem., 1995, 60, 1990-1998.]. The solution phase methyl cation affinities (MCAs) of some biological sulfur systems have been studied [Mudd, W. A., Klee P. D. Biochem, 1966, 5, 1653.]. Unfortunately, gas phase MCAs are limited to experimental and theoretical estimates for dimethylsulfide [Deakyne, C. A.; Knuth, D. M.; Meot-Ner, M.; Breneman, C. M.; Liebman, J. F. J. Mol. Struct. 1999, 485/486, 33-41] and theoretical estimates of some other sulfides [Markham, G. D. and Bock, C. W. J Phys Chem, 1993, 97,5562-5569.]. The bond strengths of other breaking alkyl groups appear to be unknown in the both the condensed phase and gas phase. There have however, been several studies on the fragmentation reactions of sulfonium ions as studied by mass spectrometry [Katritzky, A R Shipkova, P A Watson, C H Eyler, J R. and Kevill D N. Int J Mass Spectrom, 1997, 165/166, 577-583.; Buckley, N; Maltby, D; Burlingame, A L.; Oppenheimer, N J. J. Org. Chem. 1996, 61, 2753-62; (d) Mestdagh, H; Morin, N; Rolando, C. Org. Mass Spectrom. 1986, 21, 321-327.; Mestdagh, H; Morin, N; Rolando, C. Org. Mass Spectrom. 1988, 23, 246-51.]. Scheme 3 (pathway 3) is highly unlikely given that the least stable carbocation is formed (CH₃⁺). Problems associated with Scheme 3 (pathways 4 and 5) can be avoided by choosing R₁ groups which do not yield stable ions (e.g. avoid benzyl groups etc) and which do not form an alkene (e.g. avoid ethyl or substituted ethyl groups and higher homologues), respectively. Differentiation between the fragmentation reactions of sulfonium ions of methionine and S-alkyl cysteine residues (i.e., loss of CH₃SR₁ from methionine (Scheme 1) versus loss of R₁SR₂ from cysteine (Scheme 2) is straightforward. This can be achieved here by using an alkyl group R₂ which is NOT a methyl group (i.e. R₂≠CH₃). 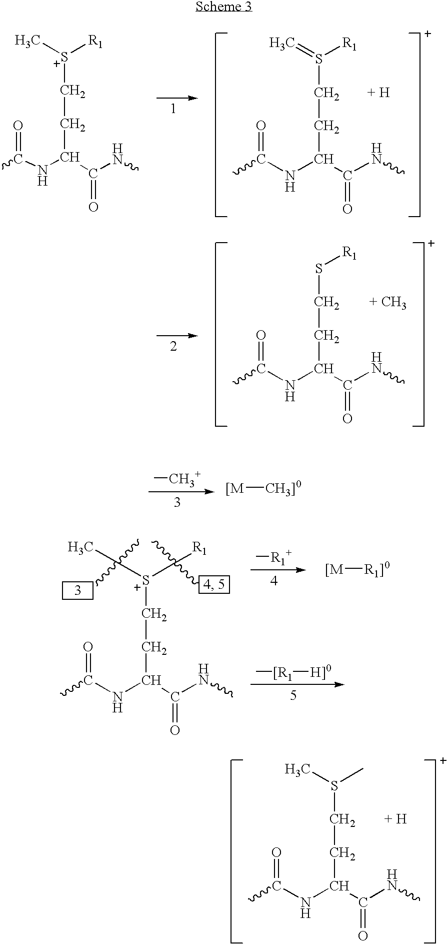
General Structure of the Sulfonium Ion Reagents Suitable for Use in the Work Described Here.

Of the reagents examined here to date, the α-carbonyl containing alkylating reagents, iodoacetic acid, iodoacetamide and bromoacetophenone are those that exhibit the favourable solution and gas phase reaction properties discussed above. Of these, acetophenone allows ready incorporation of a suitable isotopic label (for example, via synthesis of d₅-bromoacetophenone or ¹³C₆-bromoacetophenone), to enable subsequent quantitation, and has therefore been the reagent used from here on. However, any reagent having the general structure XR, where X is any suitable leaving group consisting of, for example, halides, sulfonic esters, perchlorate esters or chlorosulfonates [March J. “Advanced Organic Chemistry”, 4th Ed., Wiley, New York, 1992, pp 352-357], and R is any substrate with a carbon adjacent to the leaving group, such as alkyl, allyl or α-carbonyl groups, with the exception of those indicated above, and where an isotopic or structural label can be incorporated to allow its use for differential quantitation, would be of interest.

Examination of the Fragmentation Behaviour of Fixed-charge Sulfonium Ion Derivatives of Methionine:

CID MS/MS and MS³ in a Quadrupole Ion Trap Mass Spectrometer.

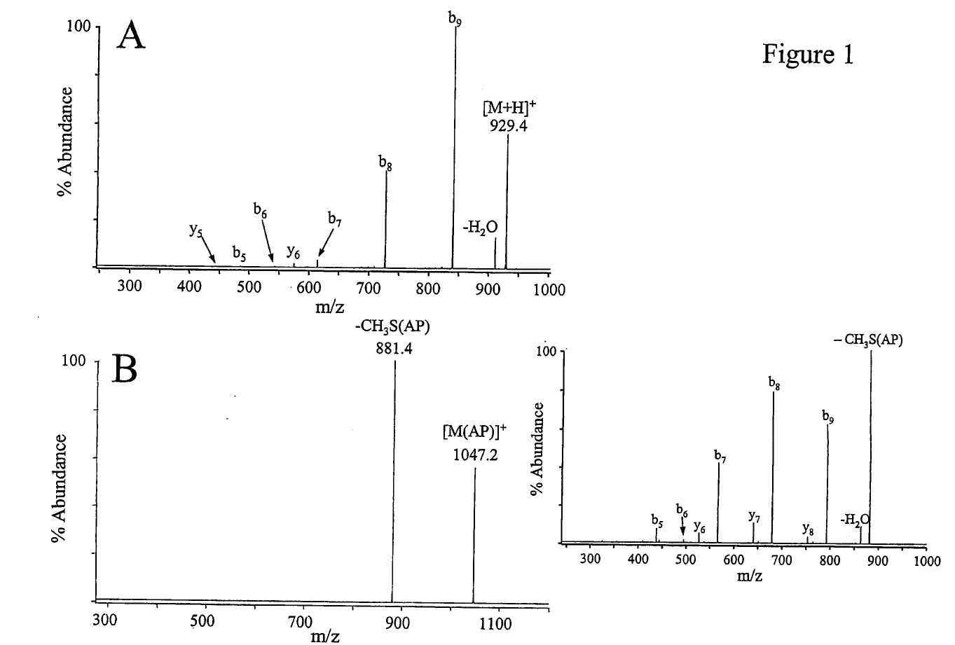

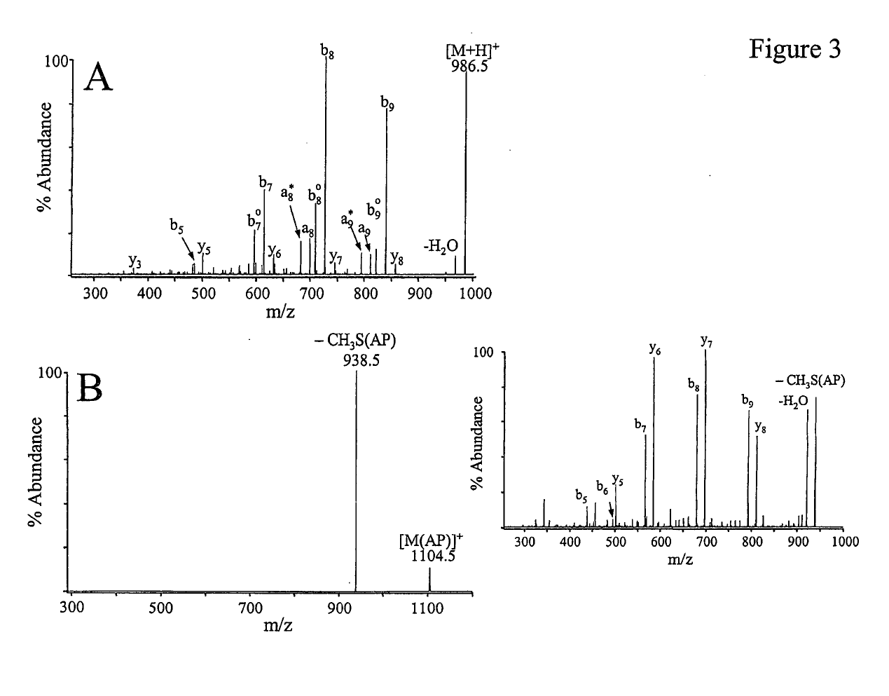

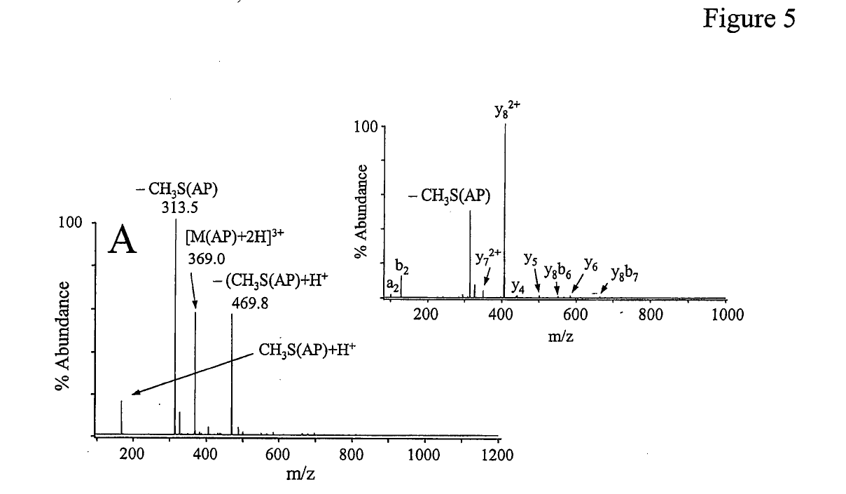

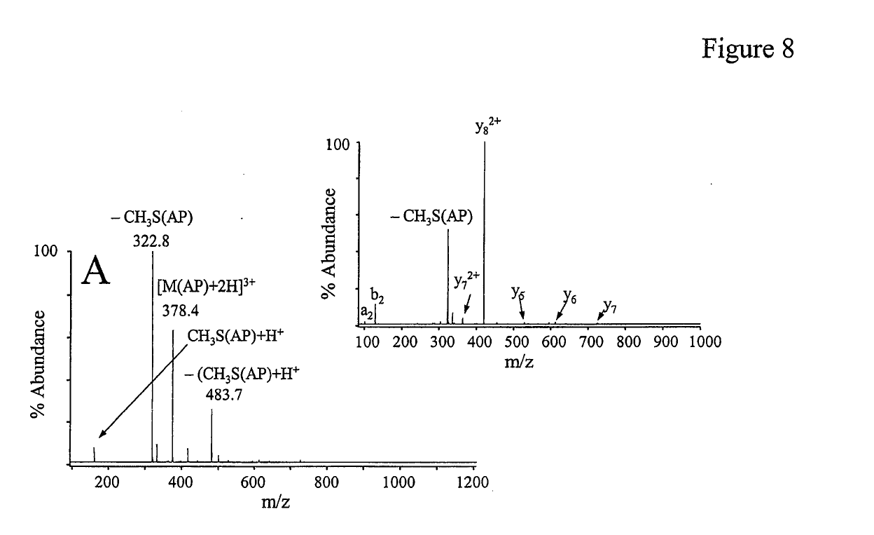

The product ion spectra obtained by CID MS/MS of the singly and doubly charged ions. of the unmodified and the acetophenone side chain derivatized sulfonium ion methionine containing peptides of GAILMGAILX (SEQ ID NO:8), where X is either alanine, lysine or arginine, and the triply charged lysine and arginine containing ions of the acetophenone side chain derivatized sulfonium ion methionine containing peptides, in a quadrupole ion trap, are shown in FIGS. 1 to 8. The singly and doubly charged forms of the unmodified peptide ions, ionized by the addition of protons only, were all observed to form product ions resulting primarily from dissociations along the peptide backbone, with b- and y-type ions dominating. In contrast, the acetophenone side chain sulfonium methionine containing peptide ion derivatives each contain a single fixed-charge resulting from the sulfonium ion, with 0, 1 or 2 protons making up the balance for the singly, doubly or triply charged ions, respectively. For these ions, in each case the overwhelmingly dominant fragmentation pathway following CID MS/MS of the isolated precursor ion corresponded to selective cleavage of the α-CH₂—S bond at the site of the fixed-charge on the methionine side chain, to yield the neutral side chain loss of S-methyl acetophenone (CH₃SCH₂COC₆H₅) (labelled as —CH₃S(AP)). Therefore, it is important to note that product ions formed by neutral loss from the side chain have the same charge state (and number of ionizing protons) as that of the precursor ion. The triply charged ion examples were the only partial exceptions, where some charged side chain loss of protonated S-methyl acetophenone (CH₃SCH₂COC₆H₅+H⁺), labeled as (—CH₃S(AP)+H⁺) in FIGS. 5A and 8A. (with the corresponding protonated S-methyl acetophenone (CH₃S(AP)+H⁺) ion also labeled), was also observed. It is expected that the charged side chain loss from the triply charged precursor ions occurs by transfer of a proton from the neutral loss product ion following dissociation of the precursor ions covalent α-CH₂—S bond, but before dissociation of the resultant ion-molecule complex to the individual products, due to columbic repulsion.

A potential mechanism, with two possible pathways for the neutral loss of CH₃SCH₂COC₆H₅ from the fixed-charge derivatives is shown in Scheme 4.

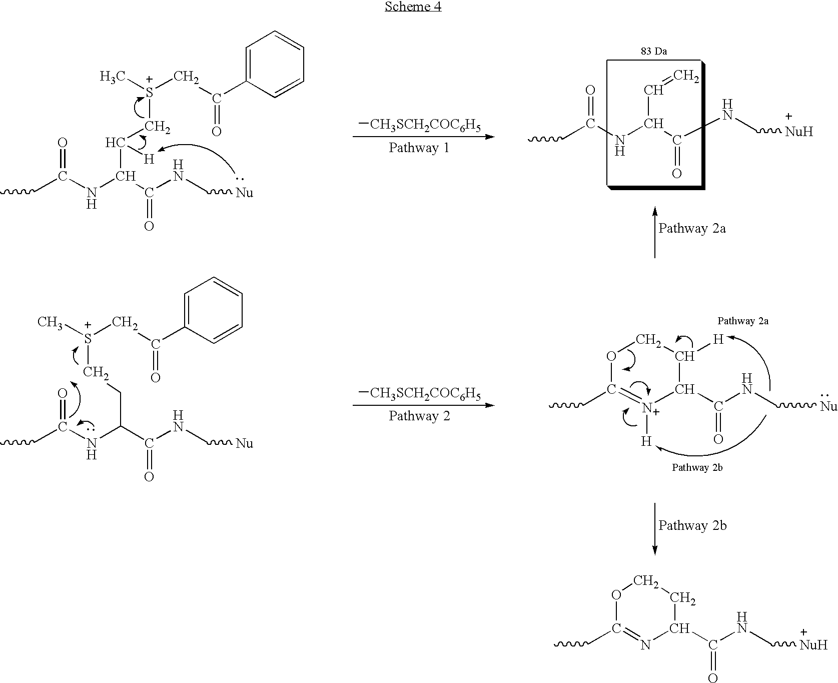

Pathway 1 of Scheme 4 involves direct proton transfer by an intramolecular nucleophile (Nu), such as the basic side chains of arginine or lysine, the N-terminal amino group or backbone amide carbonyl groups, with elimination of the neutral to yield a novel amino acid derivative, 3-amino-1-butenoic acid (vinyl glycine), with a residue mass of 83 Da Alternatively, the fragmentation may be directed by a nucleophilic attack mechanism, whereby an adjacent amide carbonyl attacks the side chain to facilitate the neutral loss, yielding a cyclic product (shown in Pathway 2 of Scheme 4 as the 6 membered cyclic product formed from nucleophilic attack by the amide carbonyl on the N-terminal side of the modified methionine residue). This mechanism is consistent with the growing recognition that nucleophilic attack processes are the major processes involved in the fragmentation of protonated peptide ions [O'Hair, R. A. J. J. Mass Spectrom. 2000, 35, 1377-1381. Schlosser, A. and Lehmann, W. D J. Mass Spectromn. 2000, 35, 1382-1390]. Intramolecular transfer of the acidic proton present in either the precursor ion (Pathway 1), or the product ion formed following methionine side chain cleavage (Pathway 2A or 2B) would result in “mobilization” of this proton, thereby allowing it to be involved in subsequent fragmentation reactions according to the mobile proton model developed for rationalizing the fragmentation of protonated peptide ions [Wysocki, V. H., Tsaprailis, G., Smith, L. L. and Breci, L. A. J. Mass Spectrom. 2000, 35, 1399-1406.]. Note that if the reaction proceeds via pathway 2 of Scheme 4, the amide bond incorporated into the cyclic product would not be amenable to subsequent cleavage. However, ring opening of the cyclic product via intramolecular transfer (Pathway 2A) would yield the acyclic amino acid derivative, thereby allowing subsequent fragmentation of the product ion to occur on both N- and C-terminal sides of the newly formed amino acid derivative.

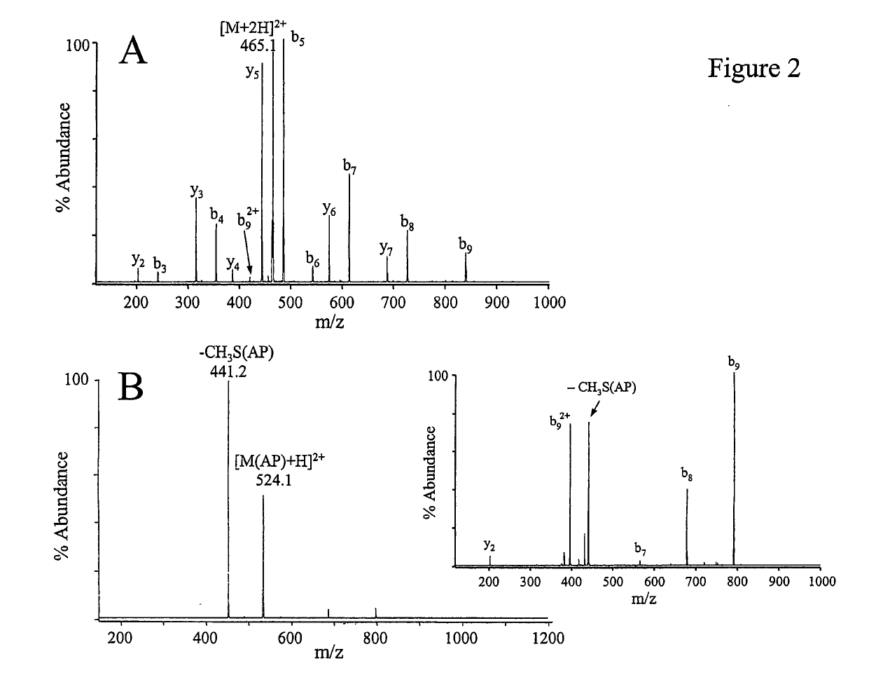

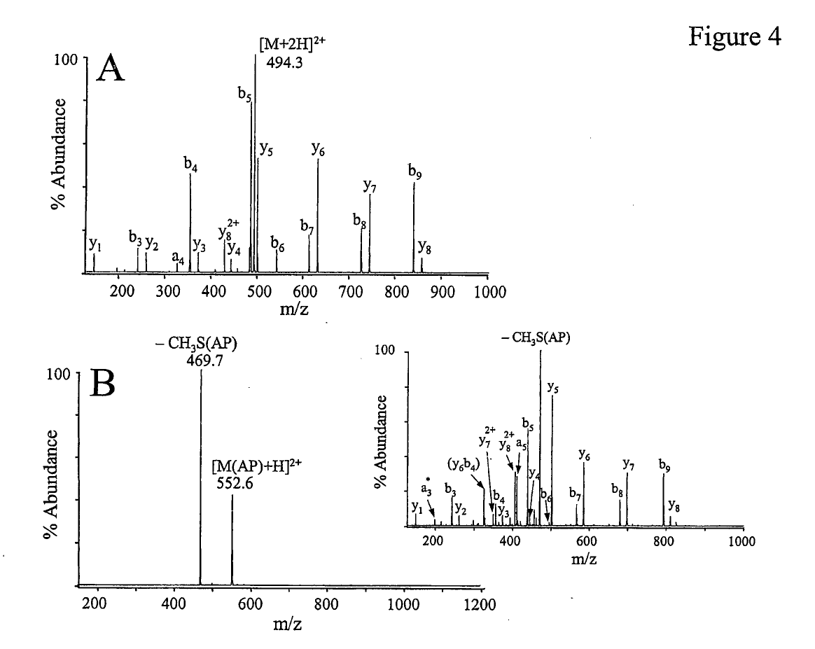

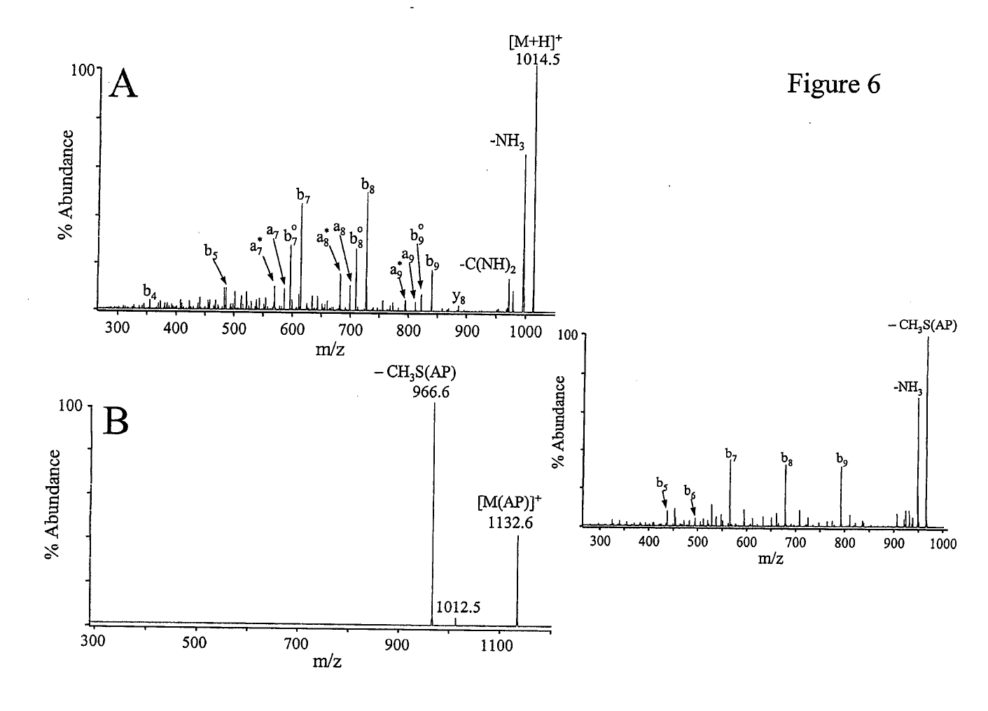

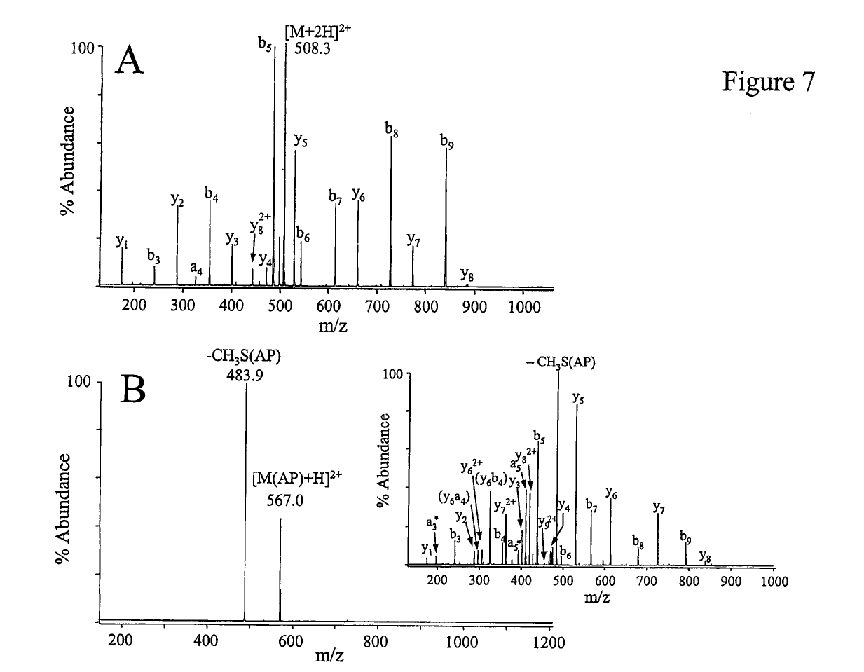

Given that the sulfonium ion derivatized peptides fragment almost exclusively via the neutral loss of CH₃SCH₂COC₆H₅ to form a charged product ion, the neutral loss product ion for each peptide ion was mass selected and then subjected to a further stage of tandem mass spectrometry (MS³) to generate additional structural information to obtain evidence for the preferred fragmentation pathway and also to allow its identification by sequence analysis. The spectra obtained following CID MS³ of each of the neutral loss product ions discussed above are shown in the insets to FIGS. 1B to 4B, the inset to FIG. 5A, the insets to FIGS. 6B and 7B, and the inset to FIG. 8A. Similar to that of the singly and doubly protonated unmodified peptide ions, MS³ of the [M+nH—CH₃SCH₂COC₆H₅]⁽ⁿ⁺¹⁾⁺ product ions formed by neutral loss from the fixed-charge derivatives resulted in the formation of products resulting primarily from dissociations along the peptide backbone, with b- and y-type ions dominating. A direct comparison of the MS/MS product ion spectra of the unmodified peptides and the MS³ spectra of the [M+nH—CH₃SCH₂COC₆H₅]⁽ⁿ⁺¹⁾⁺ product ions reveals a number of interesting points;
(i) in most cases, cleavage of the amide bonds on either side of the modified vinyl glycine residue formed by loss of the sulfonium ion side chain was observed following MS³, indicating that the fragmenting species did contain an acyclic vinyl glycine residue formed either by pathway 1 of Scheme 4 or by ring opening of the cyclic product formed by pathway 2 of Scheme 4;
(ii) for MS³ of the singly charged methionine side chain fixed-charge-peptide ions (insets to FIGS. 1B, 3B and 6B), more extensive “sequence ion” fragmentation was observed following MS³ of the [M+nH—CH₃SCH₂C₆H₅]⁽ⁿ⁺¹⁾⁺ product ions, with less “non-sequence” ion cleavages such as loss of water or ammonia and formation of a-type product ions, compared to that obtained by MS/MS of the unmodified methionine containing peptides. This observation may be readily explained by the mobile proton model for peptide fragmentation [Wysocki, V. H., Tsaprailis, G., Smith, L. L. and Breci, L. A. J. Mass Spectrom. 2000, 35, 1399-1406.]. In the case of the singly charged sulfonium ion peptide derivatives, the acidic proton formed upon loss of the side chain can be readily mobilized onto the peptide backbone by the direct proton transfer mechanism for cleavage in pathway 1 of Scheme 4, or by ring opening of the cyclic product ion shown in pathway 2A of Scheme 4, allowing subsequent fragmentation to take place at sites along the backbone, whereas, in the case of the singly protonated ions formed directly by electrospray, where the ionising proton is initially located on a basic side chain such as arginine or lysine, significant energy to overcome the barriers to proton transfer must be initially supplied in order for subsequent fragmentation of the backbone to occur. As such, other processes involving the loss of small molecules from the precursor and product ions such as water and ammonia, and formation of a-type product ions can therefore successfully compete with backbone cleavage. For the other peptides, the product ions formed by MS³ from the doubly protonated lysine and arginine containing methionine side chain fixed-charge [M+nH—CH₃SCH₂COC₆H₅]⁽ⁿ⁺¹⁾⁺ MS/MS product ions (insets to FIGS. 4B and 7B) were similar to those obtained from MS/MS of the unmodified doubly protonated peptide ions. However, MS³ of the doubly protonated product ion formed from dissociation of the acetophenone modified GAILMGAILA (SEQ ID NO:1) peptide (inset to FIG. 2B) yielded less extensive prod-cts than those formed by MS/MS of the unmodified peptide, with b₉ and b₉²⁺ ions dominating. This suggests that in this case, the proton initially present in the precursor and the acidic proton fcrmed by loss of the side chain from the doubly charged sulfonium ion derivative are not as “mobile” as those of the doubly protonated peptide where one proton is expected to reside on the amino terminal and one along the backbone; again consistent with an initial cyclic product formed from the fixed-charge derivative. The triply protonated lysine and arginine containing methionine side chain fixed-charge [M+nH—CH₃SCH₂COC₆H₅]⁽ⁿ⁺¹⁾⁺ MS³ product ions (insets to FIGS. 5A and 8A, respectively) were found to yield mainly y₈²⁺ product ions, with other y-type ions also evident at low abundance;
(iii) the collision energy required for MS³ of the [M+nH—CH₃S(AP)]⁽ⁿ⁺¹⁾⁺ product ions was consistently higher than that required for MS/MS of the unmodified peptide [M+nH]ⁿ⁺ precursor ions for each of the charge states. This suggests that the fragmentation of the precursor ions formed by neutral loss have higher activation barriers compared to the precursors formed directly by electrospray ionization, and is again suggestive of the initial formation of the cyclic product ion in pathway 2 in Scheme 4 from the fixed-charge derivatives, with a necessity for ring opening prior to subsequent further dissociation.
   Energy Resolved CID MS/MS and in a Quadrupole-time-of-flight Mass Spectrometer.

In order to determine the relative energetics for the fragmentation processes observed from the fixed-charge derivatives compared to the unmodified methionine containing peptides as well as those containing an alanine residue at the equivalent position to methionine (alanine has the closest structure of the common acids to the vinyl glycine residue formed by neutral loss from the fixed-charge derivatives and was therefore considered to be a better derivative than methionine for direct comparison of the relative energies required for fragmentation), and to obtain evidence for the structure of the resultant product ions, the fragmentation reactions of the sulfonium ion containing peptides described above were further examined by energy resolved CID in a quadrupole-time-of-flight (Q-TOF) mass spectrometer.

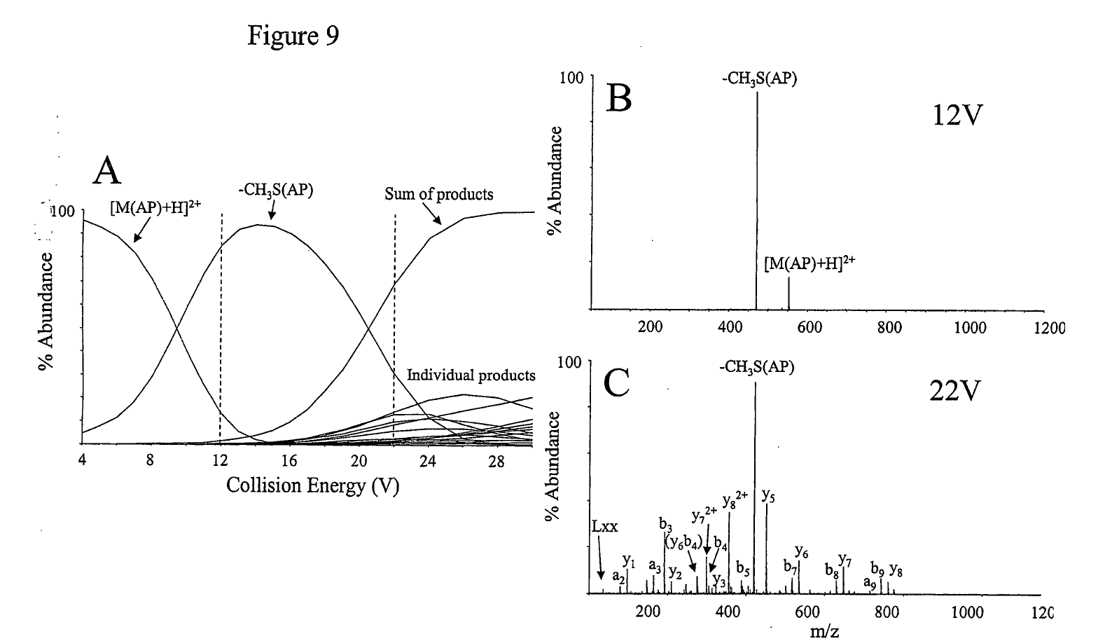

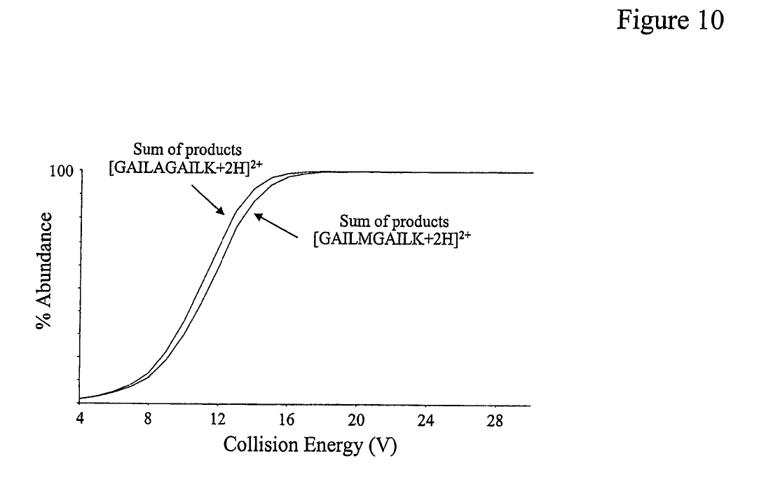

The energy resolved CID breakdown and appearance curves for the doubly charged sulfonium ion derivative of GAILMGAILK (SEQ ID NO:2) is shown in FIG. 9A. The breakdown curve of the precursor [M(AP)+H]²⁺ ion, the appearance/breakdown curve of the initial neutral loss product ion (—CH₃S(AP)), as well as the appearance curves of the individual product ions and that of the summed product ion abundances (sum of product ions) are indicated. It can be seen that there is essentially no overlap between the breakdown of the [M(AP)+H]²⁺ ion and the appearance of the individual product ions, indicating that these ions are not formed directly from the initial precursor. Rather, these ions are formed from the initial neutral loss product ion (—CH₃S(AP)). Representative product ion spectra taken under collision energies sufficient to obtain 70-90% reduction of the precursor ion abundance (12 eV) and 70-90% reduction of the initial neutral loss (—CH₃S(AP) product ion abundance (22 eV) are shown in FIGS. 9B and C, respectively. The observed product ion spectra under these “low” and “high” energy quadrupole CID conditions, respectively, were essentially identical to those observed in the quadrupole ion trap mass spectrometry MS/MS and MS³ experiments, indicating that the time frame (millisecond versus microsecond time scales for the ion trap and quadrupole experiments, respectively), and mode of ion activation had no appreciable effect on the fragmentation process. Indeed, all of the peptides examined under these “low” and “high” energy quadrupole CID conditions exhibited very similar product ion spectra compared to the equivalent ion trap derived MS/MS and MS³ spectra (data not shown). Thus, the directed fragmentation of the fixed-charge derivatives gives the ability to use low energy CID conditions for formation of the initial neutral loss product ion and then to obtain a “pseudo MS³” spectrum under “high” energy CID conditions to obtain further structural information. For comparison, the appearance curves for the formation of the summed product ion abundances from the doubly protonated unmodified methionine and alanine containing peptides are shown in FIG. 10.

A summary of the collision energy required to achieve 50% formation of product ions from each of the protonated unmodified peptides, and from the initial neutral loss product ions from the fixed-charge derivatives is given in Table 1. These data can be used as a measure of the stabilities of the sulfonium ion derivative neutral loss product ions relative to their unmodified forms. The data indicates that the collision energies required for dissociation of the initial neutral loss product ions from the sulfonium ion derivatives are consistently higher than those of the alanine and umnodified methionine containing peptides, giving further support for the formation of an initial cyclic product ion which is required to overcome a barrier to ring opening prior to subsequent dissociation (Scheme 4, pathway 2A).

TABLE 1

Collision Energy required to achieve 50% reduction in
the abundance of the unmodified precursor ions, or the
initial neutral loss product ions from the fixed-
charge sulfonium ion derivatives, in a Q-TOF mass
spectrometer.

Peptide Ion Charge State

| | | |
|---|---|---|
| +1 | +2 | +3 |

| | |
|---|---|
| Peptide | Collision Energy (V) |

| | | | |
|---|---|---|---|
| GAILAGAILA (SEQ ID NO:4) | 21.7 | 8.1 | NA |
| GAILMGAILA (SEQ ID NO:1) | 22.7 (+1.0)^{a} | 8.6 (+0.5)^{a} | NA |
| GAILM(AP)GAILA^{b} (SEQ ID NO: 1) | 34.5 (+12.8)^{a} | 14.8 (+6.7)^{a} | NA |
| GAILAGAILK (SEQ ID NO:5) | 36.8 | 10.8 | NA |
| GAILMGAILK (SEQ ID NO:2) | 38.4 (+1.6)^{a} | 11.4 (+0.6)^{a} | 11.4 |
| GAILM(AP)GAILK^{b} (SEQ ID NO:2) | 43.0 (+6.2)^{a} | 21.5 (+10.7)^{a} | 11.6 |
| GAILAGAILR (SEQ ID NO:6) | 47.1 | 10.8 | NA |
| GAILMGAILR (SEQ ID NO:3) | 48.6 (+1.5)^{a} | 11.6 (+0.8)^{a} | NA |
| GAILM(AP)GAILR^{b} (SEQ ID NO:3) | 53.7 (+6.6)^{a} | 22.1 (+11.3)^{a} | 11.7 |

^{a}Difference between the collision energy required for 50% dissociation of the peptide of interest and its corresponding unmodified alanine containing peptide ion.
^{b}(AP) = acetophenone side chain modification to methionine

Note that an alternative to performing an additional stage of CID MS/MS to determine the identities of those peptides found to contain a fixed charge derivative, would be the use of high resolution mass accuracies (an “accurate mass tag”) coupled with database searching. Previously, in cases where an “accurate mass tag” approaching 1 ppm has been obtained, and the presence of a particular aniino acid is known (for example the presence of a cysteine residue), the specificity of database searching algorithms can be improved such that unambiguous identification of the protein from which the peptide is derived has been achieved from this information alone [Conrads, T. P., Anderson, G. A., Veenstra, T. D., Pasa-Tolic, L. and Smith, R. D. Anal. Chem. 2000, 72, 3349-3354.; Goodlet, D. R., Bruce, J. E., Anderson, G. A., Rist, B., Pasa-Tolic, L., Fiehn, O., Smith, R. D. and Aebersold, R. Anal. Chem. 2000, 72, 1112-1118.; Strittmatter, E. F., Ferguson, P. L., Tang, K. and Smith, R. D. J. Am. Soc. Mass Spectrom. 2003, 14, 980-991.].

Identification of Peptides Containing Methionine by Neutral Loss Scan Mode CID MS/MS of Their Sulfonium Ion Fixed-charge Derivatives in a Triple Quadrupole Mass Spectrometer.

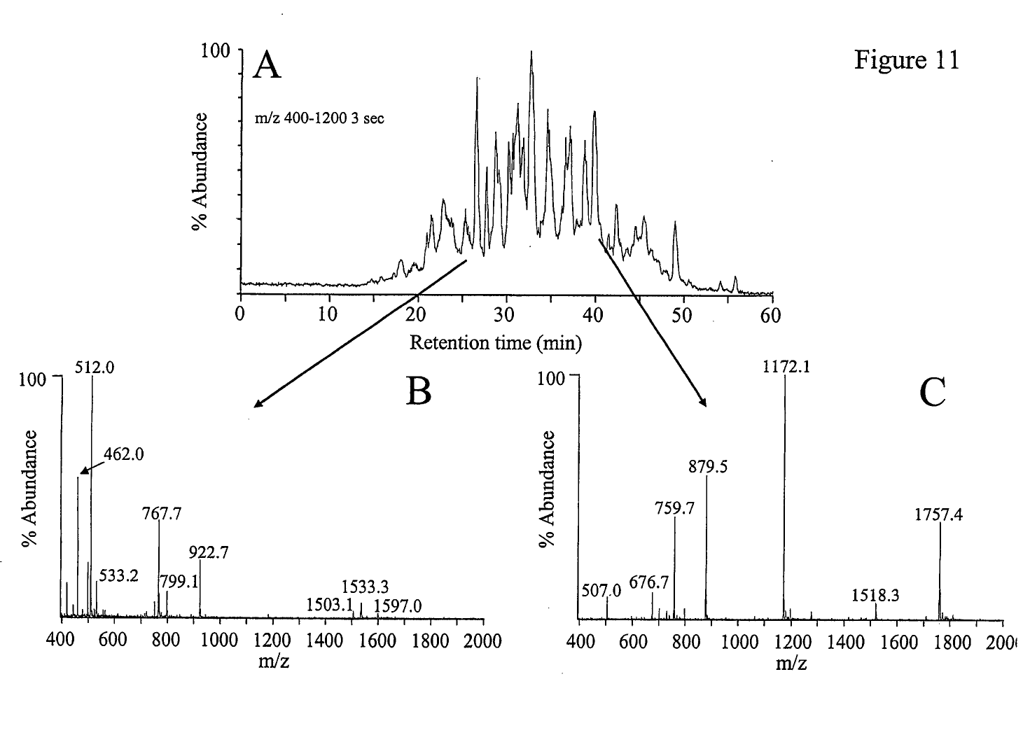

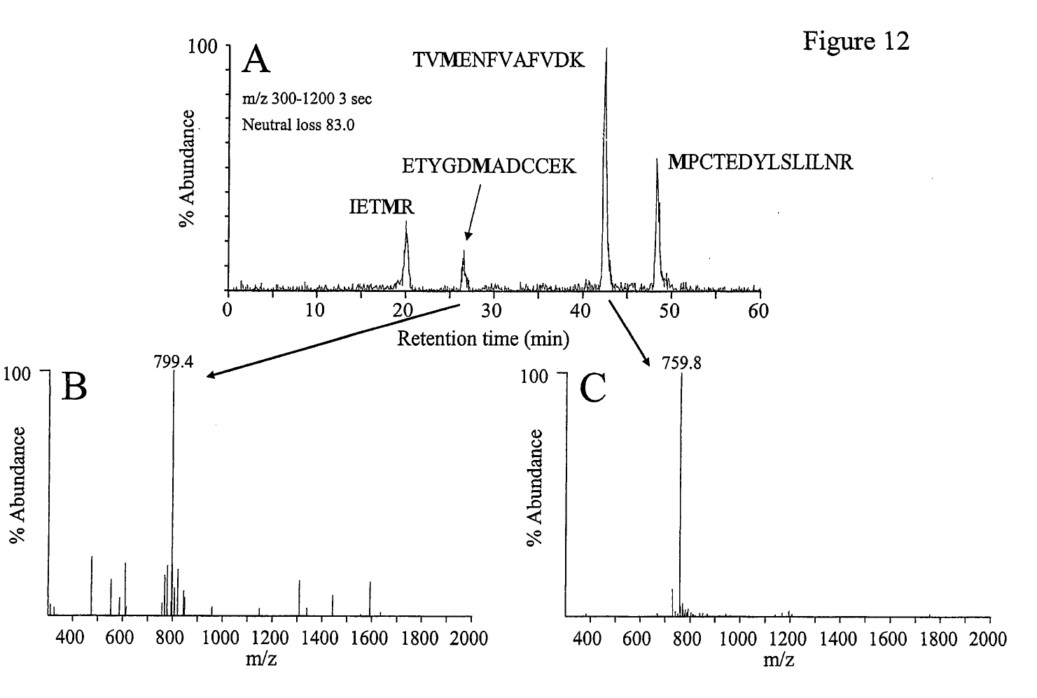

To demonstrate the utility of the fixed charge derivatives for selectively identifying peptides containing certain amino acid resides in a relatively complex mixture by neutral loss scan mode CID MS/MS, 10 pmol of a reduced and S-carboxyamidomethylated tryptic digest of bovine serum albumin, derivatized using bromoacetophenone, was subjected to capillary RP-HPLC-MS (FIG. 11). Then, in a duplicate experiment, those peptides giving rise to a neutral loss of m/z 83 (i.e., the loss of CH₃SCH₂COC₆H₅ from doubly charged precursor ions) were then identified in a neutral loss scan mode CID MS/MS experiment. The resultant total ion current trace for this experiment is shown in FIG. 12A. Four main peaks were observed and the mass spectra obtained from two of these are shown in FIGS. 12B and 12C. The masses of these peaks were found to correspond to the four methionine containing tryptic peptides of bovine serum albumin. It can be observed by comparison of the data in FIGS. 11B and C (the mass spectra of the two regions of the total ion current chromatogram from the MS experiment indicated by arrows), and FIGS. 12B and C, that the ions detected in the neutral loss scan mode experiment are present at relatively low abundance (the ion at m/z 799.4 in FIG. 12B is only the 8^{th} most abundant ion in FIG. 11B, and the ion at m/z 759.8 in FIG. 12C is only the 3d most abundant ion in FIG. 11C). Therefore, these data demonstrate the utility of the fixed charge derivatives for selectively identifying peptides containing certain amino acid resides by neutral loss scan mode CID MS/MS of their fixed charge derivatives (the four methionine containing tryptic peptides of bovine serum albumin were selectively identified from a total of approximately 100 peptides present in the digest).

Strategies for Protein Identification by Neutral Loss Scan Mode CID MS/MS of Peptides Containing Other Amino Acid Fixed-charge Derivatives.

Tryptophan and Cysteine

Modification of tryptophan, by electrophilic aromatic substitution, to yield a fixed-charge sulfonium ion derivative could be achieved by initial reaction of tryptophan containing peptides with a sulfenylhalide (R₁SX) to yield a derivative with a thioether functional group in the 2 position of the indole side chain (Scheme 5A) [Scoffone, E., Fontana, A. and Rochhi, R. Biochemistry. 1968, 7, 971.], followed by the formation of the sulfonium ion derivative of tryptophan under conditions identical to those discussed above for methionine and S-alkyl cysteine sulfonium ion derivatives. Note that the sulfenylhalide would also react with cysteine containing peptides to yield an unsymmetrical disulfide (Scheme 5B), however, specificity of the reaction to tryptophan may be imparted by initial S-alkylation of cysteine, or reduction of the unsymmetrical disulfide prior to sulfonium ion formation. The solution phase and gas phase criteria for sulfonium ion formation from 2-indole S-alkyl tryptophan modified residues are the same as those discussed above for methionine and for S-alkyl cysteine sulfonium ion formation. As sulfonium ion derivatives of the 2-indole S-alkyl derivatives of tryptophan will be formed under conditions identical to those discussed above for methionine and S-alkyl cysteine, both residues would be modified in this approach. However, for the reactions discussed above for methionine and S-alkyl cysteine sulfonium ion derivatives, methionine and tryptophan containing peptides could be identified individually, through the use of an R₁ group that does not correspond to CH₃, (i.e., the side chain alllyl group of methionine) or simultaneously, via initial alkylation of tryptophan where the R₁ group corresponds to CH₃. Likewise, 2-indole S-alkyl tryptophan and S-alkyl cysteine containing peptides could be identified individually, through the introduction of an R₁ group to the S-alkyl tryptophan derivative that does not match that of the R₁ group used for initial alkylation of cysteine, or simultaneously, via initial alkylation of tryptophan using the same R₁ group as that used for initial cysteine alkylation. Note that under appropriate conditions, similar electrophilic aromatic substitution reactions could also be used for the introduction of an S-alkyl group to the side chain of tyrosine residues, followed by subsequent formation of a sulfonium ion derivative. 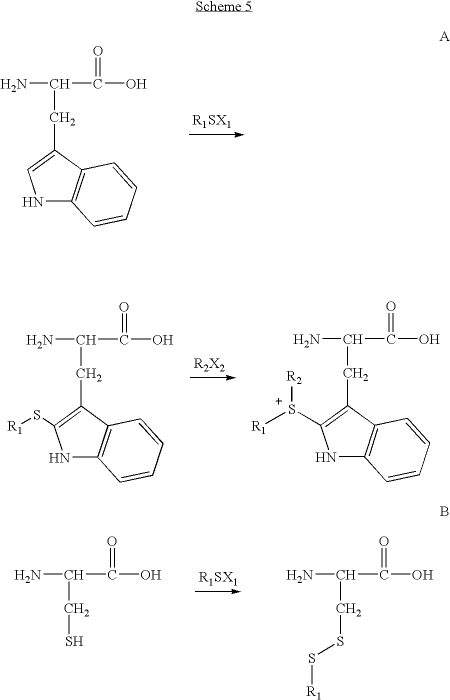
General Structure of the Fixed-charge Ion Reagents Suitable for Use in the Work Described Here.

Any reagent having the general structure R₁SX₁, where X₁ is any suitable leaving group consisting of, for example, halides, sulfonic esters, perchlorate esters or chlorosulfonates [March J. “Advanced Organic Chemistry”, 4th Ed., Wiley, New York, 1992, pp 352-357], and R₁ is any substrate with a carbon adjacent to the leaving group, such as alkyl, allyl or α-carbonyl groups, with the exception of those indicated above, and where an isotopic or structural label can be incorporated to allow its use for differential quantitation, and R₂X₂, where X₂ is any suitable leaving group consisting of, for example, halides, sulfonic esters, perchlorate esters or chlorosulfonates [March J. “Advanced Organic Chemistry”, 4th Ed., Wiley, New York, 1992, pp 352-357], and R₂ is any substrate with a carbon adjacent to the leaving group, such as alkyl, allyl or α-carbonyl groups, with the exception of those indicated above, and where an isotopic or structural label can be incorporated to allow its use for differential quantitation.

Sulfonium ions of tryptophan and cysteine containing peptides may also be formed directly, by initial reaction with a sulfenylhalide (X₂RSX₁) containing a suitable leaving group X₂, to subsequently yield a cyclic sulfonium ion derivative of tryptophan (Scheme 6A) or cysteine (Scheme 6B). 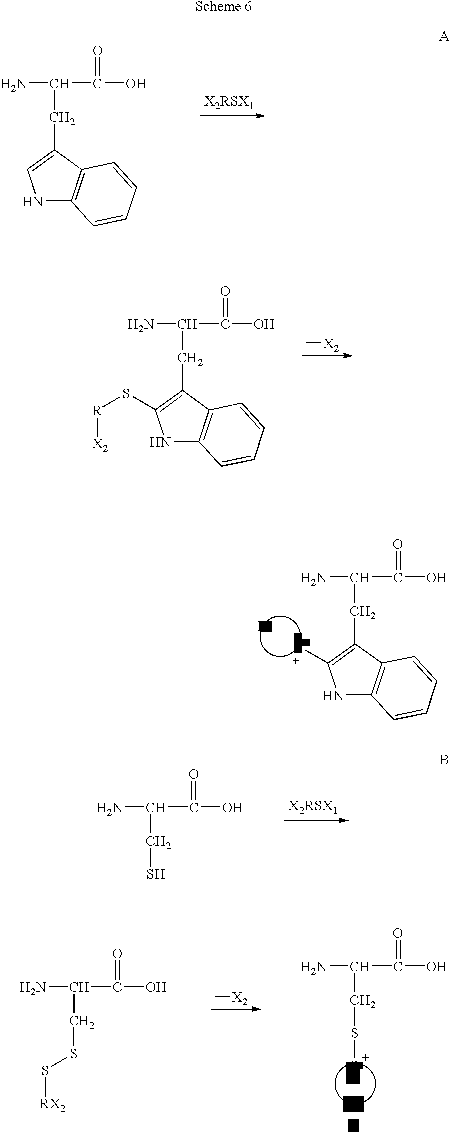
General Structure of the Fixed-charge Ion Reagents Suitable for Use in the Work Described Here.

Any reagent having the general structure X₂RSX₁, where X₁ is any suitable leaving group consisting of, for example, halides, sulfonic esters, perchlorate esters or chlorosulfonates [March S. “Advanced Organic Chemistry”, 4th Ed., Wiley, New York, 1992, pp 352-357], R is any substrate with a carbon adjacent to the leaving group, such as alkyl, allyl or α-carbonyl groups, with the exception of those indicated above, and where an isotopic or structural label can be incorporated to allow its use for differential quantitation, and X₂ is any suitable leaving group consisting of, for example, halidesi sulfonic esters, perchlorate esters or chlorosulfonates [March J. “Advanced Organic Chermistry”, 4th Ed., Wiley, New York, 1992, pp 352-357].

Cysteine.

Cyclic sulfonium ions may also be formed specifically from cysteine containing peptides, by initial alkylation with a reagent (X₂RX₁) (Scheme 7A) or by initial disulfide exchange with a reagent (X₂RSSX₁) (Scheme 7B), each containing a suitable leaving group X₂, with subsequent formation of a cyclic sulfonium ion derivative. 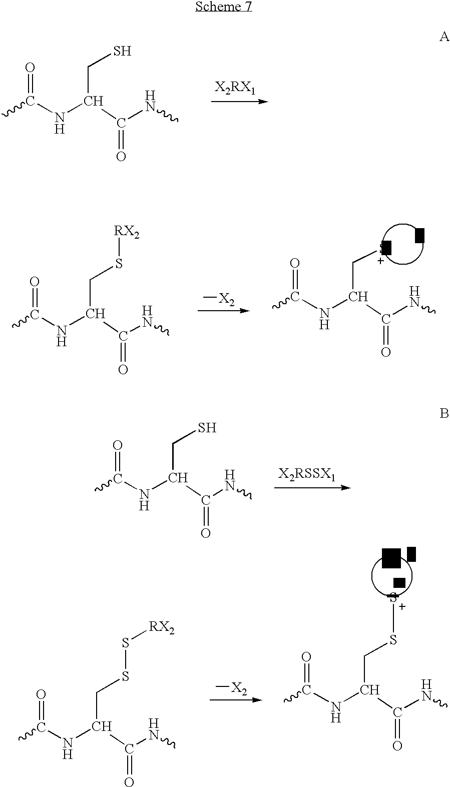
General Structure of the Fixed-charge Ion Reagents Suitable for Use in the Work Described Here.

Any reagent having the general structure (i) X₂RX₁, where X₁ is any suitable leaving group consisting of, for example, halides, sulfonic esters, perchlorate esters or chlorosulfonates [March J. “Advanced Organic Chemistry”, 4th Ed., Wiley, New York, 1992, pp 352-357], R is any substrate with a carbon adjacent to the leaving group, such as alkyl, allyl or α-carbonyl groups, with the exception of those indicated above, and where an isotopic or structural label can be incorporated to allow its use for differential quantitation, and X₂ is any suitable leaving group consisting of, for example, halides, sulfonic esters, perchlorate esters or chlorosulfonates under neutral or basic pH conditions, or protonated amines or alcohols under acidic pH conditions [March J. “Advanced Organic Chemistry”, 4th Ed., Wiley, New York, 1992, pp 352-357], or (ii) X₂RSSX₁, where X₁ is, for example, nitrobenzoic acid, R is any substrate with a carbon adjacent to the leaving group, such as alkyl, allyl or α-carbonyl groups, with the exception of those indicated above, and where an isotopic or structural label can be incorporated to allow its use for differential quantitation, and X₂ is any suitable leaving group consisting of, for example, halides, sulfonic esters, perchlorate esters or chlorosulfonates [March J. “Advanced Organic Chemistry”, 4th Ed., Wiley, New York, 1992, pp 352-357].

Strategies for Protein Identification by Precursor Ion Scan Mode CID MS/MS of Peptides Containing Other Amino Acid Fixed-charge Derivatives.

In contrast to the fragmentation reactions discussed above, where neutral losses are observed (note that the site of the fixed charge in these derivatives is directly adjacent to the site of bond cleavage), the fragmentation of peptides derivatized on their side chains where the fixed charge is located remote to the site of bond cleavage, may result in the loss of a charged product, which may be selectively detected using a precursor ion scan mode MS/MS experiment (Scheme 8). For example, fixed-charge sulfonium, quaternary alkylammonium or quaternary alkylphosphonium ion reagents should result in charged loss of the side chain via cis 1,2 elimination, with formation of a dominant low mass product ion with formation of its complementary neutral peptide species (if the initial charge state of the precursor was one), or a charged peptide ion species with a charge state one lower than the precursor (if the initial charge state of the precursor was greater than one). These characteristic low mass product ion(s) can be selectively detected using a parent ion scan mode MS/MS experiment. This side chain cleavage may be further enhanced by oxidation of the thioether [Steen, H. and Mann, M. J. Am. Soc. Mass Spectrom. 2002, 13, 996-1003.]. Note that the fragmentation of cysteine-containing peptides alkylated with reagents containing basic functionalities, such as iodoacetamide, acrylamide and vinylpyridine, have been observed previously to give rise to some fragmentation of the side chain [Moritz, R. L. Eddes, J. S., Reid, G. E. and Simpson, R. J. Electrophoresis. 1995, 17, 907-917.].

The solution phase and gas phase requirements for quaternary alkylammonium or quaternary alkylphosphonium ion formation and fragmentation are the same as those for sulfonium ion formation and fragmentation discussed above. Note that a fixed-charge derivative of cysteine (using (2-bromoethyl)trimethylammonium bromide (BrCH₂CH₂(CH₃)₃NBr) for introduction of the fixed charge to the side chain thiol of the cysteine residue), has been used previously to enable carboxypeptidase C-terminal peptide sequence analysis, with mass spectrometry used for detection of the reaction products [Bonetto, V., Bergman, A-C., Jornvall, H. and Sillard, R. Anal. Chem. 1997, 69, 1315-1319.]. 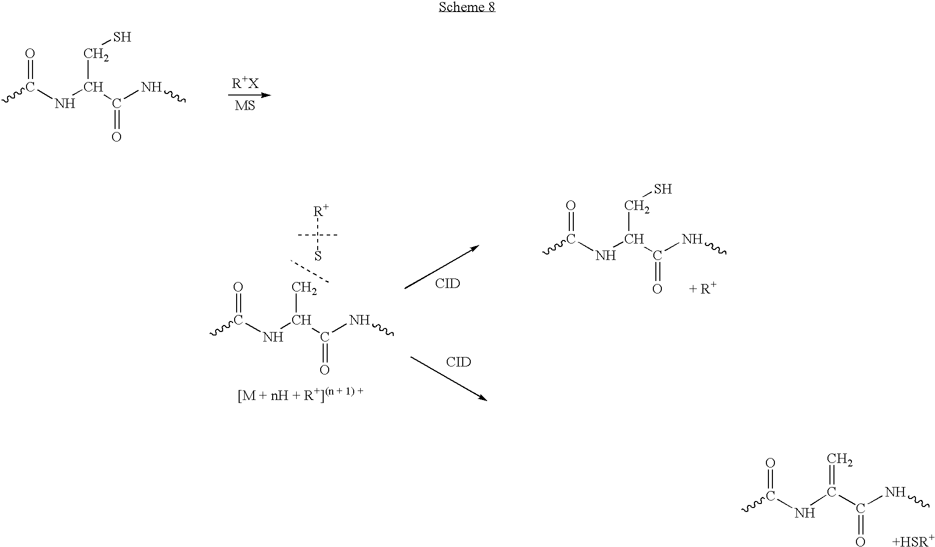
General Structure of the Fixed-charge Ion Reagents Suitable for Use in the Work Described Here.

Any reagent having the general structure R+X, where X is any suitable leaving group consisting of, for example, halides, sulfonic esters, perchlorate esters or chlososulfonates [March J. “Advanced Organic Chemistry”, 4th Ed., Wiley, New York, 1992, pp 352-357], and R⁺ is any substrate with a carbon adjacent to the leaving group, such as alkyl, allyl or α-carbonyl groups, and with a terminal sulfonium, quaternary alkylammonium or quaternary alkylphosphonium ion, and where an isotopic or structural label can be incorporated to allow its use for differential quantitation.

Other Mass Spectrometry Instrumentation and Ionization Source Considerations.

It is important to recognise that the dissociation of fixed charge derivatives with neutral loss of the side chain, results in a peptide product ion having the same charge state (and number of ionising protons) as that of the precursor ion. Hence, these derivatives are amenable to both ESI (where multiply charged ions are usually observed) and MALDI (where predominantly singly charged ions are observed) ionization methods when identification of the peptide sequence is to be achieved by “high” energy MS/MS or MS³. In contrast, the dissociation of fixed charge derivatives with charged loss of the side chain (i.e., formation of a low mass product ion), results in a peptide product that carries one less charge than that of the precursor ion. Therefore, if identification of the peptide sequence is to be achieved by further dissociation, this approach is only applicable when using ESI methods, where a charged product ion containing the peptide would be formed in addition to the charged side chain cleavage low mass product ion.

Differential Quantitation of Proteins by CID MS/MS of Their Fixed-charge Derivative Containing Peptides.

Neutral Loss Scan Mode CID MS/MS in a Triple Quadrupole Mass Spectrometer.

In a general strategy for differential quantitation of protein expression using the fixed-charge derivatization approach described here, a first sample is derivatized with either sulfonium or tertiary alkyl fixed-charge reagents containing naturally abundant (light) isotopes, then mixed with a second sample derivatized with the same reagent containing isotopically enriched (heavy) isotopes, prior to their introduction to the mass spectrometer. Then, using either neutral loss or parent ion scan mode MS/MS, the abundance ratios of the product ions formed by neutral loss or parent ion scan mode MS/MS of the light isotope tag containing peptide ions, compared to those containing the heavy isotope tag can be determined, which is indicative of changes in the abundance levels of proteins between the two samples. The product ion representing the peptide product can then be automatically selected for further structural interrogation by “high” energy MS/MS or MS³. Note that the approach described here could also be readily combined with a “global” labelling (i.e., N- or C-terminal labelling) strategy, whereby the isotopic label is introduced at a position remote to the site of the fixed-charge so that a common neutral loss could be used to identify both light and heavy isotope containing derivatives.

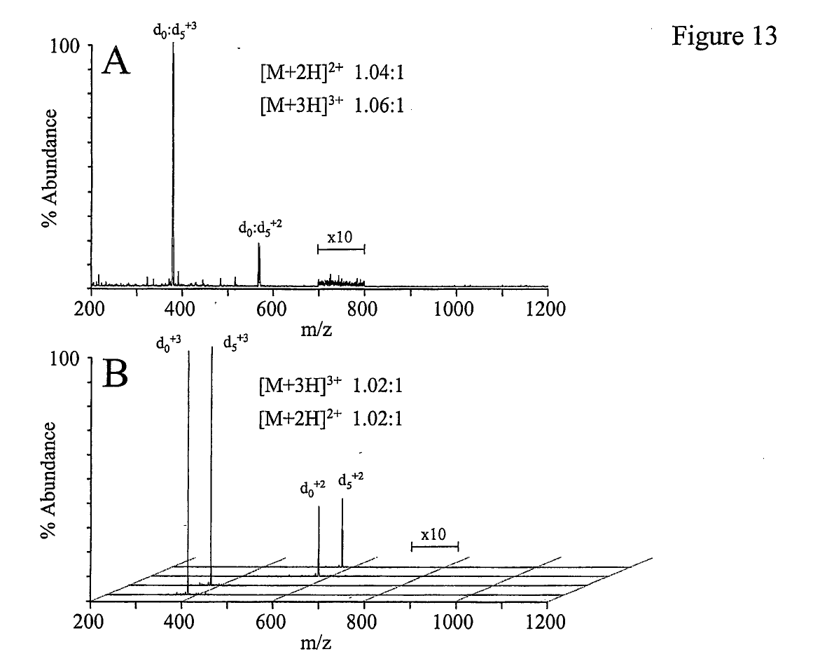

The ESI mass spectrum of a nominal 1.0 pmol/μL: 1.0 pmol/μL mixture of the GAILMGAILR (SEQ ID NO:3) peptide derivatized with either d₀- or d₅-acetophenone and introduced by electrospray ionization to a triple quadrupole mass spectrometer is shown in FIG. 13A. From this mass spectrum, a ratio of 1.04:1 can be obtained for the doubly (m/z 567.2 and 569.7) charged, and 1.06:1 for the triply (m/z 378.4 and 380.1) charged states of the peptide. A 10 times expanded region of the spectrum around m/z 700-800 shows the level of chemical noise associated with the mass spectrum. In comparison, the neutral loss scan mode MS/MS spectra of the same mixture (neutral losses of 83 Da and 85.5 Da, respectively for the doubly charged precursors, and neutral losses of 55.3 Da and 57 Da, respectively for the triply charged precursors) are shown in FIG. 13B. Again, the m/z 700-800 regions for each spectrum have been expanded 10 times to indicate the level of chemical noise. While the observed ratios for the MS/MS neutral loss scan mode ion abundances in FIG. 13B were essentially identical to those observed for the MS ion abundances in FIG. 13A (a ratio of 1.02:1 was obtained for both doubly and triply charged states of the peptide), the level of chemical noise is reduced by at least two orders of magnitude compared to that observed in the mass spectrum in FIG. 13A. Indeed, with the exception of electronic noise, characterised by single data point noise “spikes”, the neutral loss MS/MS spectra showed a complete absence of non-specific chemical noise along the baseline.

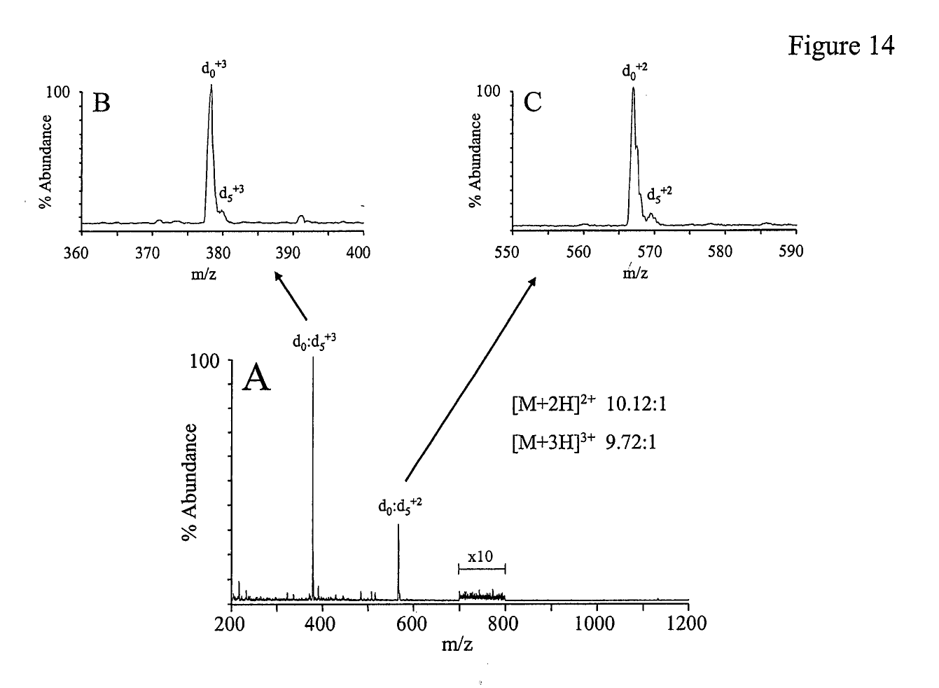

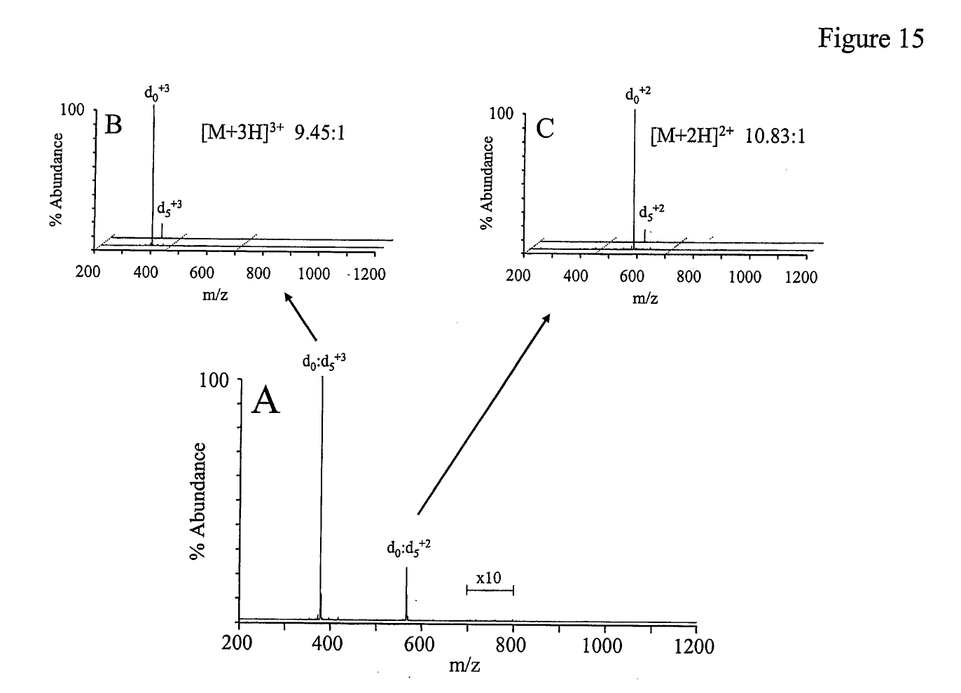

The mass spectrum of a nominal 1 pmol/μL: 0.1 pmol/μL mixture of d₀- and d₅-containing methionine side chain fixed-charge acetophenone (AP) sulfonium ion derivative of GAILMGAILR (SEQ ID NO:3) is shown in FIG. 14. The abundance ratios obtained for the doubly and triply charged ions were determined to be 10.12:1 and 9.72:1, respectively. The ratios of the ions obtained by neutral loss scan mode MS/MS experiments (FIG. 15) were determined to be 9.45:1 and 10.83:1. Once again, the level of chemical noise in the neutral loss scan mode experiment was found to be at least two orders of magnitude lower compared to that observed in the mass spectrum. For all neutral loss scan mode MS/MS experiments, (ratios of 0.1:1, 0.2:1.0, 0.5:1.0, 1.0:1.0, 1.0:0.5, 1.0:0.2 and 1.0:0.1), errors were all within 10% of the expected values.

Note that for the neutral loss product ions formed from the sulfonium ion derivatized peptide precursors, a common product is formed from both “heavy” and “light” ions, therefore either product may be selected for further structural analysis by “high” energy CHD or MS³.

One of the additional advantages of the MS/MS method described here over those involving identification by MS peak abundances alone is that both ions (i.e., light and heavy) are not required to be present in order for detection of one of the pairs to be achieved. Also, in these instances, the mass of the neutral loss from the parent ion indicates its origin (i.e., from the “light” or “heavy” sample).

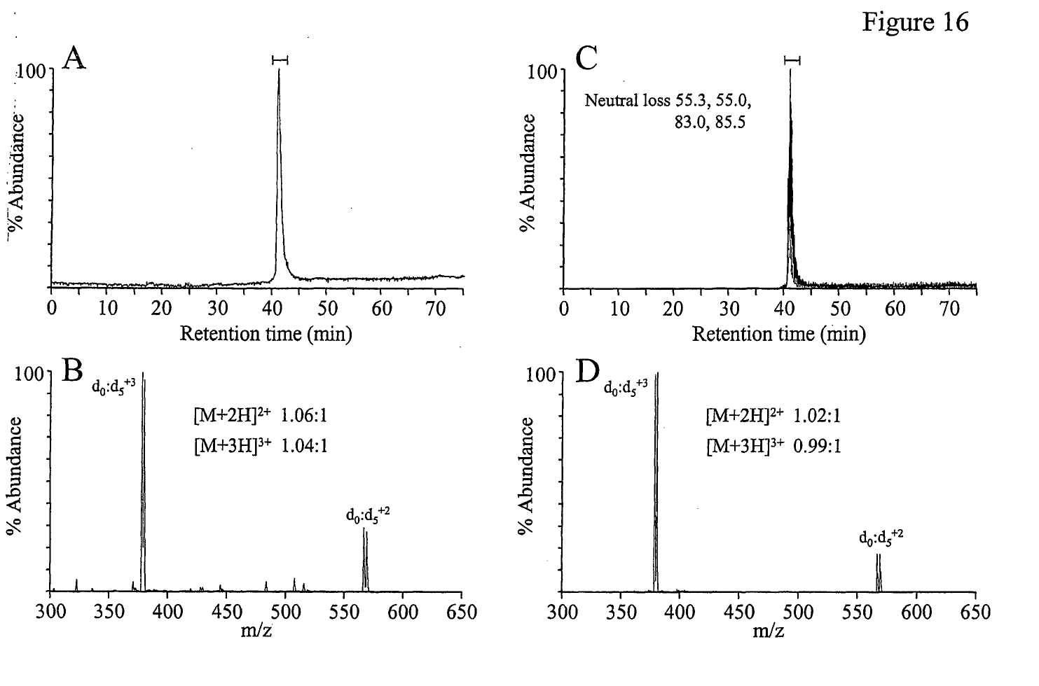

To demonstrate the utility of the isotopically labelled fixed charge derivatization approach coupled with neutral loss scan mode MS/MS for differential quantitation of peptides containing certain amino acid resides, 1 pmol each of d₀- and d₅-acetophenone derivatized GAILMGAILR (SEQ ID NO:3) peptide was subjected to capillary RP-BPLC-MS and neutral loss scan mode CID MS/MS (FIGS. 16A and 16C, respectively). The resultant mass spectra obtained from the MS (ratios of 1.06:1 and 1.04:1 for the doubly and triply charged ions) and neutral loss MS/MS scans (ratios of 1.02:1 and 0.99:1 for the doubly and triply charged ions) are shown in FIGS. 16B and 16C. The neutral loss scans for 83 Da and 85.5 Da for the doubly charged d₀- and d₅-labelled precursors, and 55.3 Da and 57 Da for the triply charged d₀- and d₅-labelled precursors have all been summed. Less than 10% resolution of the d₀- and d₅-forms of the fixed charge derivatives was observed, indicating that there is not a strong deuterium isotope effect on the chromatographic separation for these fixed charge derivatives. This is consistent with recent reports on the factors controlling deuterium isotope effects on chromatography [Zhang, R.; Sioma, C. S.; Wang, S.; Regnier, F. E. Anal. Chem. 2001, 73, 5142-5149.; Zhang, R., Sioma, C. S.; Thompson, R. A.; Xiong, L.; Regnier, F. E. Anal. Chem. 2002, 74, 3662-3669.; Zhang, R. and Regnier, F. E. J. Proteome Res. 2002, 1, 139-147.

Identification and Differential Quantitation of O-linked Post Translationally Modified Proteins by CID MS/MS or Ion-ion Reaction MS/MS of Their Peptides Containing Fixed-charge Derivatives:

If combined with the β-elimination/Michael addition chemistry for forming mass spectrometry stable derivatives of serine and threonine O-linked post translationally modified amino acid containing peptides, the fixed-charge derivatization approach could be extended toward the identification and quantitation of O-linked post translational modification status in proteins. Following β-elimination of O-linker serine or threonine post translational modifications, a one or two step Michael addition process could be used for fixed-charge introduction. Scheme 9 illustrates a one step process whereby the fixed-charge derivative (for example, a quaternary ammonium or phosphonium ion derivative) is introduced directly. Alternatively, in a two step process, a neutral side chain derivative could be introduced (e.g., an alkyl thiol), followed by subsequent derivatization to form a fixed charge sulfonium ion. Therefore, sulfonium, quaternary alkylammonium, or quaternary alkylphosphonium ion chemistries as outlined above, could also be used for selective detection of peptides containing the modified amino acid residue. A similar method has been described by Steen and Mann for the introduction of a dimethylamine-containing sulfenic acid derivative to β-eliminated phospho-serine and phospho-threonine residues, thereby allowing a characteristic protonated low mass fragment ion at m/z 122.06 to be detected by precursor ion scan mode MS/MS upon low energy CID (Steen, H. and Mann, M. J. Am. Soc. Mass Spectrom. 2002, 13, 996-1003). However, this ion is not always observed as a dominant product as the formation of this ion can be competitive with backbone fragmentation processes. Therefore, a mixture of product ions without and with the modified side chain can be present simultaneously, resulting in a complex product ion spectrum, thereby making it potentially difficult to interpret without prior knowledge of the expected peptide sequence. The fixed charge derivatization method described here however, would result in the exclusive loss of the modified side chain, allowing improved detection of these peptides at higher levels of sensitivity and with better control of the subsequent dissociation process for subsequent structural elucidation. When combined with the differential isotopic labelling strategy outlined above, the fixed charge derivatization method of the present invention would also allow differential quantitation of the O-linked post translational modification status. 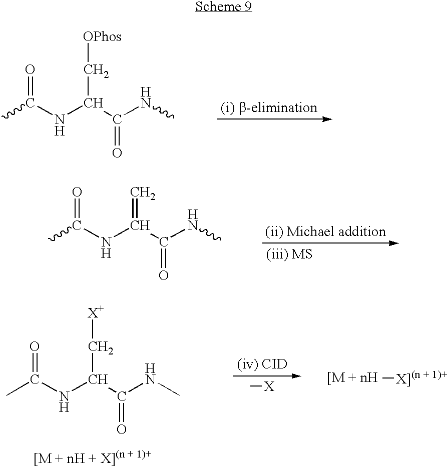
Characterization of Protein-Protein Interactions by CID MS/MS or Ion-ion Reaction MS/MS Identification of Cross-linked Peptides Containing Fixed-charge Derivatives.

The fixed-charge derivatization approach could also be extended toward the improved characterization of protein-protein interactions by incorporation of the fixed-charge derivative into a suitable cross-linking reagent prior to cross-linking reactions, or by deritivatization of a cross-link contained between two proteins or peptides after a cross-linking reaction which, upon CID MS/MS, fragments via the specific loss of a neutral or charged entity from the cross-link. While the incorporation of a labile MS/MS “tag” on a cross linker has been used by Back et al. to identify cross linked peptides via the detection of a characteristic low mass product ion formed upon low energy CID [Back, J. W., Hartog, A. F., Dekker, H. L., Muijsers, A. O., de Koning, L. J. and de Jong, L. J. Am. Soc. Mass Spectrom. 2001, 12, 222-227.], the product ion thus formed is seen as only a relatively low abundance product ion. In contrast, in a similar manner to that employed for peptide identification and quantitation using fixed-charge derivatives as outlined above, the method of the present invention could be employed here to direct the fragmentation of cross linked peptides toward exclusive cleavage of the fixed-charge, to yield either a characteristic neutral loss or low mass product ion (Scheme 10). The product ion containing the cross linked peptide may then be automatically selected for further structural interrogation by higher energy MS/MS or by MS³. If quantitative comparison of cross linking between two different samples was required, an isotopic label could be also incorporated into the cross-link.

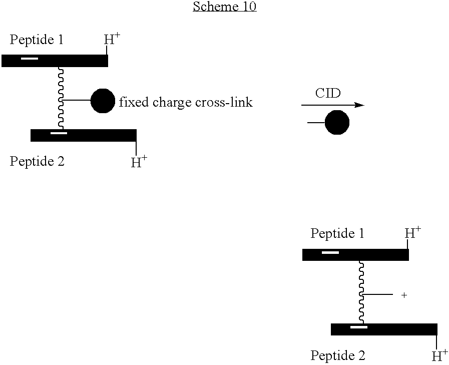

If desired, any of the examples discussed above containing fixed-charge derivatives could also be selectively pre-enriched prior to mass spectrometric analysis by known chromatographic methods [Tang, J. R. and Hartley, B. S. Biochem. J. 1967, 102, 593.; Degen, J. and Kyte, J. Anal. Biochem. 1978, 89, 529-539.; Kyte, J., Degen, J. and Harkins, R. N. Methods Enzymology 1983, 91, 367-377.; Gevaert, K., Van Damme, J., Goethals, M., Thomas, G. R., Hoorelbeke, B., Demol, H., Martens, L., Puype, M., Staes, A. and Vandekerckhove, J. Mol. Cell. Proteomics. 2002, 1, 896-903.].

Alternatively the fixed-charge derivatives could be selectively pre-enriched by solid phase capture methods, using fixed charge reagents covalently coupled to beads or insoluble polymers [Weinberger, S. R.; Viner, R. I. and Ho, P. Electrophoresis. 2002, 23, 3182-3192.; Zhou, H.; Ranish, J. A.; Watts, J. D.; Aebersold, R. Nature Biotechnol. 2002, 20, 512-515.; Qiu, Y; Sousa, E. A.; Hewick, R. M.; Wang, J. H. Anal. Chem. 2002, 74, 4969-4979.; Qian, W-J, Goshe, M. B., Camp D. G., Yu, L-R, Tang, K. and Smith. R. D. Anal. Chem. 2003, 75, 5441-5450.]. Chemical or photolytic cleavage would then be employed to release the fixed-charge derivative of the peptide (shown for a sulfonium ion fixed charge derivative in Scheme 11), thereby reducing the sample complexity of the mixture to be analysed prior to its introduction to the mass spectrometer.

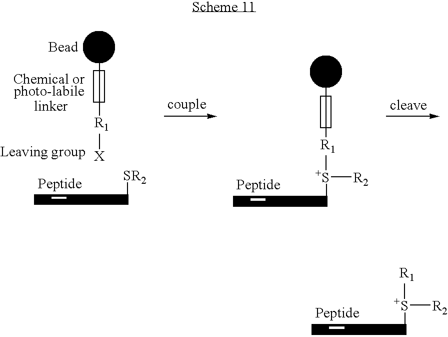

### Preferred Embodiments for Methionine Fixed Charge Derivatization Reagents

The two structures immediately shown below are those that have been used in the work described here to date. These are bromoacetophenone and the isotopically encoded deuterated (d₅) version.

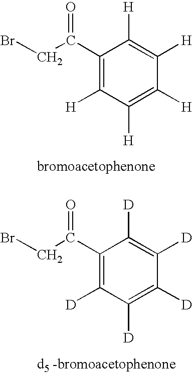

A preferred isotopically encoded reagent of this form is a ¹³C₆ labelled version, whose structure is shown below. ¹³C labelling is preferred over deuterium labelling in order to minimise any chromatographic separation of the isotopically labelled versus unlabelled reagents. This reagent would be expected by those of skill in the art to have the same properties as the unlabelled form.

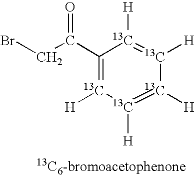

A more preferred reagent is a water soluble form of the foregoing reagent, as this added property confers benefits such as allowing for more streamlined sample preparation workflows. Examples of water soluble forms of the reagents are shown below, as well as their ¹³C₆ labelled versions. Note that the addition of a sulfinic or sulfonic acid functional group could be achieved at the ortho, meta- or para positions relative to the acetyl group. The synthetic strategy can be chosen to direct the synthesis toward the any one of these positions; the para substituted position is shown below.

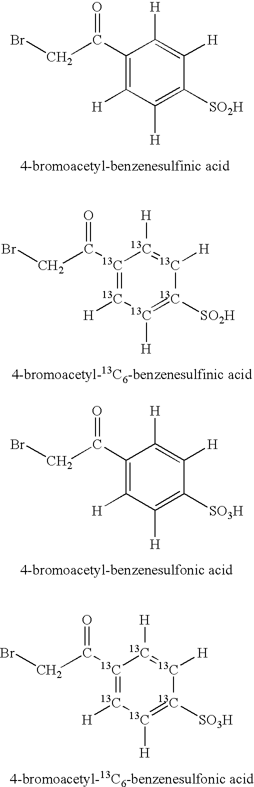

A non-limiting method for the synthesis of these derivatives may be obtained using methods known to those skilled in the art [Buess, C. M. and Kharasch, N. J. Am. Chem. Soc. 1950, 72, 3529-3532.; Kharasch, N. and Swidler, R. J. Org. Chem. 1954, 19, 1704-1707.; Vogels Textbook of Practical Organic Chemistry. 4th ed. 1978. p 773-778.; Szmant, H. H. and Irwin, D. A. J. Am. Chem. Soc. 1956, 78, 4386-4389.] via (i) reaction of benzene with 2,4-dinitrophenylsulfenyl chloride to yield phenyl 2,4-dinitrophenyl sulfide, (ii) further reaction with acetic anhydride to yield the p-acetylbenzene 2,4-dinitrophenyl sulfide (iii) oxidization to yield the p-acetylbenzene 2,4-dinitrophenyl sulfone (iv) cleaved with methanolic alkali to yield the p-acetylbenzene 2,4-dinitrophenyl sulfinic acid, then either (v) further oxidised to the sulfonic acid and then brominated or (v) directly brominated.

Alternatively, following steps (i) and (ii), the derivative may be (iii) cleaved by thiolysis to yield the p-acetylthiobenzene derivative (iv) oxidised to the sulfinic or sulfonic acids, then (v) brominated.

While a bromine atom is shown as the leaving group (i.e., X, of RX) in the foregoing structures, an iodine also preferred. Other leaving groups that could be used include: halides such as a chlorine atom, as well as sulfonic esters, perchlorate esters, chlorosulfonates, or protonated amines or alcohols [March J. “Advanced Organic Chemistry”, 4th Ed., Wiley, New York, 1992, pp 352-357].

In addition to the free acids shown above, these compounds may be prepared in their salt forms (e.g., Na or HCl), and can be prepared as solvates.

The invention also includes reagent kits for analysis of amino acids, peptides or proteins by mass spectrometry. The reagent kits include one or more of the foregoing compounds and other reagents such as protein disulfide reducing agents (e.g., dithiothreitol (DTT), β-mercaptoethanol, tris-carboxyethyl phosphine (TCEP), tributylphosphine (TBP)), cysteine alkylating reagents (e.g., alkylhalides (e.g., iodoacetic acid, iodoacetamide) as well as vinylpyridine or acrylamide), proteases or chemical cleavage agents (e.g., trypsin, Lys-C, Asp-N, pepsin, thermolysin, cyanogen bromide, dilute acid). The reagent kits also can contain solvents. Typical solvents that are used include urea, guanidine hydrochloride, acetonitrile, methanol, water.

The various reagents can be used to perform the foregoing methods steps in solution, or on a solid phase, (e.g., by having the reagents immobilized on a column).

Amino acids or peptides labelled using the method of the invention can be used, in addition to analysis of amino acids, peptides and proteins, for internal standards. For example, the derivatized peptides described herein can be used in a manner analogous to several recent reports that used known quantities of an isotopically encoded peptide derivative spiked into a sample as an internal standard in order to perform absolute quantitation of the selected peptide of interest (see Gygi, Steven P.; Peng, Jumnin. PCT Int. Appl. (2003) WO 2003078962; Gerber, Scott A.; Rush, John; Stemman, Olaf; Kirschner, Marc W.; Gygi, Steven P. Proc. Natl. Acad. Sci. U.S. A. (2003), 100, 6940-6945.). Note that these reports do not employ the use of fixed charge derivatives amenable to tandem mass spectrometry detection as are describe herein.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The disclosures of the references cited herein are hereby incorporated herein by reference.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of analysis of amino acids, peptides or proteins, the method comprising:
(1) derivatizing a mixture of amino acids, peptides or proteins, to form at least one amino acid, peptide or protein derivatized to contain a fixed-charge ion, other than at the C-terminal or N-terminal end thereof;
(2) introducing the mixture of amino acids, peptides or proteins containing at least one amino acid, peptide or protein derivatized to contain a fixed-charge ion other than at the C-terminal or N-terminal end thereof, to a mass spectrometer;
(3) passing the mixture of amino acids, peptides or proteins containing at least one amino acid, peptide or protein derivatized to contain a fixed-charge ion, other than at the C-terminal or N-terminal end thereof, through a first mass resolving spectrometer to select precursor protein or peptide ions having a first mass-to-charge ratio;
(4) subjecting the precursor ions of the first mass-to-charge ratio to dissociation to form product ions having a second mass-to-charge ratio that is characteristic of a fragmentation occurring at a site adjacent to the fixed charge; and
(5) detecting the product ions having the second mass-to-charge ratio.

2. The method of claim 1, wherein the product ions having the second mass-to-charge ratio are product ions formed by neutral loss of the fixed charge from the precursor ions.

3. The method of claim 1, wherein the product ion having the second mass-to-charge ratio are product ions formed by charged loss of the fixed charge from the precursor ions.

4. The method of claim 1, further comprising the step of:
(6) determining the identity of the derivatized peptide or protein.

5. The method of claim 4, wherein the step of determining the identity of the derivatized peptide or protein is performed by first repeating steps (1), (2), (3) and (4) and then subjecting the product ions having the second mass-to-charge ratio to dissociation to form a series of product ions having a range of mass-to-charge ratios, for the purpose of determining the amino acid sequence of the peptide or protein.

6. The method of claim 5, wherein the product ions having the second mass-to-charge ratio are formed by neutral loss from the precursor.

7. The method of claim 5, wherein the wherein the product ion having the second mass-to-charge ratio is formed by charged loss from the precursor ion.

8. The method of claim 4, wherein the step of determining the identity of the derivatized peptide or protein is performed by use of high resolution mass analyzers.

9. The method of claim 8, wherein the use of high resolution mass analyzers provides mass accuracies of approximately 1-5 ppm on the product ion detected in step (5), or its complementary product ion.

10. The method of claim 4, wherein the step of determining the identity of the derivatized peptide or protein comprises database searching to identify those peptides found to contain a fixed charge derivative.

11. The method of claim 1, wherein the step of dissociation comprises a method selected from the group consisting of (i) collisions with an inert gas (collision-induced dissociation (CID) or collisionally-activated dissociation (CAD)); (ii) collisions with a surface (surface-induced dissociation (SID)); (iii) interaction with photons resulting in photodissociation, optionally using a laser; (iv) thermal/black body infrared radiative dissociation (BIRD); and (v) interaction with an electron beam, resulting in electron-induced dissociation for singly charged cations (EID), electron-capture dissociation (ECD) for multiply charged cations, or combinations thereof.

12. The method of claim 1, wherein the method is used for identification of amino acids, peptides or proteins.

13. The method of claim 1, wherein the method is used for quantitation of amino acids, peptides or proteins.

14. The method of claim 1, wherein the method is used for amino acid, peptide or protein differential quantitation based on the incorporation of suitable isotopic or structural labels to the fixed charge.

15. The method of claim 14, wherein the isotopic labels are selected from the group consisting of ¹³C, ¹⁵N, and ²H.

16. The method of claim 1, wherein the method is used for analysis of post translational modification status of amino acids, peptides or proteins.

17. The method of claim 16, wherein the analysis of post translational modification status of amino acids, peptides or proteins comprises incorporation of the fixed-charge derivative via a P-elimination/Michael addition method for forming mass spectrometry stable derivatives of O-phosphorylated and O-glycosylated serine, or O-phosphorylated and O-glycosylated threonine.

18. The method of claim 1, wherein the method is used for analysis of cross-linking status of amino acids, peptides or proteins.

19. The method of claim 1, wherein the method is used for analysis of interaction of proteins.

20. The method of claim 1, wherein the fixed-charge derivative is contained on the side-chain of a selected amino acid residue or a side-chain of a selected amino acid residue contained within a protein or peptide.

21. The method of claim 20, wherein the selected amino acid residue is that of a rare amino acid.

22. The method of claim 20, wherein the selected amino acid residue contains a S atom.

23. The method of claim 22, wherein the amino acid residue is methionine, cysteine, homocysteine or selenocysteine.

24. The method of claim 23, wherein the amino acid residue is methionine and wherein the method is performed according to any of Schemes 1, 3 and 4.

25. The method of claim 23, wherein the amino acid residue is cysteine and wherein the method is performed according to any of Schemes 2, 5, 6, 7 and 8.

26. The method of claim 20, wherein the selected amino acid residue is tryptophan or tyrosine.

27. The method of claim 26, wherein the amino acid residue is tyrosine or tryptophan and wherein the method is performed according to any of Schemes 5 and 6.

28. The method of claim 20, wherein the amino acid contains an S-alkyl group.

29. The method of claim 28, wherein the amino acid residue is methionine, S-alkyl cysteine, S-alkyl homocysteine, S-alkyl tryptophan or S-alkyl tyrosine.

30. The method of claim 1, wherein the fixed-charge derivative is contained on a side-chain of a post-translationally modified amino acid residue.

31. The method of claim 30, wherein the fixed-charge derivative is contained on an O-linked post-translationally modified amino acid residue.

32. The method of claim 30, wherein the O-linked post-translationally modified amino acid residue is a dehydroalanine residue formed by β-elimination from an O-linked post-translationally modified serine amino acid residue (Scheme 9).

33. The method of claim 30, wherein the O-linked post-translationally modified amino acid residue is a dehydroamino-2-butyric acid residue formed by β-elimination from an O-linked post-translationally modified threonine amino acid residue.

34. The method of claim 1, wherein the fixed-charge derivative is contained on a cross-link contained between two amino acids, peptides or proteins (Scheme 10).

35. The method of claim 1, wherein the fixed-charge derivative is contained within a cross-linking reagent.

36. The method of claim 1, wherein the fixed-charge derivative is selectively pre-enriched by solid phase capture methods using fixed charge reagents covalently coupled to beads or insoluble polymers (Scheme 11).

37. The method of claim 1, wherein the fixed-charge ion is a sulfonium ion, a quaternary alkylammonium or a quaternary alkylphosphonium ion.

38. The method of claim 1, wherein the analysis of the amino acid, peptide or protein ion is performed by tandem mass spectrometry.

39. The method of claim 38, wherein the tandem mass spectrometer is equipped with electrospray ionization (ESI) or matrix assisted laser desorption ionization (MALDI) interfaces to transfer the protein or peptide ion into the gas-phase.

40. The method of claim 38, wherein the tandem mass spectrometer is a tandem-in-space mass spectrometer, a tandem-in-time mass spectrometer, or a combination thereof.

41. The method of claim 40, wherein the tandem-in-space mass spectrometer is a sector mass spectrometer, a time of flight mass spectrometer, a triple quadrupole mass spectrometer, or a hybrid mass spectrometer combining time of flight and quadrupole instruments.

42. The method of claim 41, wherein the sector mass spectrometer is a double focusing sector mass spectrometer or a hybrid mass spectrometer combining sector and quadrupole instruments.

43. The method of claim 38, wherein the tandem-in-time mass spectrometer is a two-dimensional quadrupole ion trap mass spectrometer, a three-dimensional quadrupole ion trap mass spectrometer or a Fourier-transform ion cyclotron resonance (FT-ICR) mass spectrometer.

44. The method of claim 1, further comprising one or more steps of protein extraction, protein separation, reduction and alkylation of cysteine disulfides ander digestion.

45. The method of claim 1, wherein the amino acids, peptides or proteins are derivatized using a substituted acetophenone, or a salt thereof, or a solvate thereof, having the following formula:

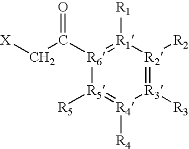

46. The method of claim 45, wherein X is any halogen, sulfonic ester, perchlorate ester or chlorosulfonate.

47. The method of claim 45, wherein R₁-R₅ are H, and R₁′-R₆′ are ¹²C.

48. The method of claim 45, wherein the substituted acetophenone is an isotopically encoded substituted acetophenone, or a salt thereof, or a solvate thereof.

49. The method of claim 48, wherein X is any halogen, sulfonic ester, perchlorate ester or chlorosulfonate.

50. The method of claim 49, wherein at least one of, and preferably at least three of R₁-R₅ are 2H, and R₁′-R₆′ are ¹²C.

51. The method of claim 49, wherein R₁-R₅ are H, and at least one of, and preferably at least three of R₁′-R₆′ are ¹³C.

52. The method of claim 45, wherein at least one of R₁ -R₅ is a functional group containing an atom other than hydrogen or carbon.

53. The method of claim 45, wherein the substituted acetophenone is water soluble.

54. The method of claim 53, wherein
R₁ is SO₂H, and R₂-R₅ are H, and R₁′-P6′ are ¹²C;
R₁ is H, R₂ is SO₂H, and R₃-R₅ are H, and R₁′-R₆′ are ¹²C;
R₁₋₂ are H, R₃ is SO₂H, and R₄-R₅ are H, and R₁′-R₆′ are ¹²C;
R₁ is SO₃H, and R₂-R₅ are H, and R₁′-R₆′ are ¹²C;
R₁ is H, R₂ is SO₃H, and R₃-R₅ are H, and R₁′-R₆′ are ¹²C; or
R₁₋₂ are H, R₃ is SO₃H, and R₄-R₅ are H, and R₁′-R₆′ are ¹²C.

55. The method of claim 53, wherein
R₁ is SO₂H, and at least one of, and preferably at least three of R₂-R₅ are ²H, and R₁′-R₆′ are ¹²C;
R₁ is SO₂H, and R₂-R₅ are H, and at least one of, and preferably at least three of R₁′-R₆′ are ¹³C;
R₂ is SO₂H, and at least one of, and preferably at least three of R₁ and R₃-R₅ are ²H, and R₁′-R₆′ are ¹²C;
R₂ is SO₂H, and R₁ and R₃-R₅ are H, and at least one of, and preferably at least three of R₁′-R₆′ are ¹³C;
R₃ is SO₂H, and at least one of, and preferably at least three of R₁ -R₂ and R₄-R₅ are ²H, and R₁′-R₆′ are ¹²C;
R₃ is SO₂H, and R₁-R₂ and R₄-R₅ are H, and at least one of, and preferably at least three of R₁′-R₆′ are ¹³C;
R₁ is SO₃H, and at least one of, and preferably at least three of R₂-R₅ are ²H, and R₁′-R₆′ are ¹²C;
R₁ is SO₃H, and R₂-R₅ are H, and at least one of, and preferably at least three of R₁′-R₆′ are ¹³C;
R₂ is SO₃H, and at least one of, and preferably at least three of R₁ and R₃-R₅ are 2H, and R₁′-R₆′ are ¹²C;
R₂ is SO₃H, and R₁ and R₃-R₅ are H, and at least one of, and preferably at least three of R₁′-R₆′ are ¹³C;
R₃ is SO₃H, and at least one of, and preferably at least three of R₁-R₂ and R₄-R₅ are 2H, and R₁′-R₆′ are ¹²C; and
R₃ is SO₃H, and R₁-R₂ and R₄-R₅ are H, and at least one of, and preferably at least three of R₁′-R₆′ are ¹³C.

56. A substituted acetophenone, or a salt thereof, or a solvate thereof, having the following formula: 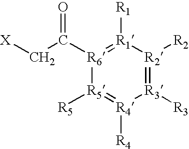
wherein X is a sulfonic ester, perchlorate ester or chlorosulfonate. R₁-R₅ are H and R₁′-R₆′ are selected from the group consisting of ¹²C and ¹³C, or R₁-R₅are ²H and R₁′-R₆′ are ¹³C.

57. (canceled)

58. The substituted acetophenone of claim 56, wherein the substituted acetophenone is an isotopically encoded substituted acetophenone, or a salt thereof, or a solvate thereof.

59-60. (canceled)

61. A water soluble substituted acetophenone, or a salt thereof, or a solvate thereof, having the following formula: 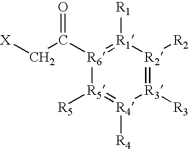
wherein X is Br or I, R₁-R₅ are H and R₁′-R₆′ are selected from the group consisting of ¹²C and ¹³C or R₁-R₅ are ²H and R₁′-R₆′ are ¹³C.

62. (canceled)

63. The water soluble substituted acetophenone of claim 61, wherein X is Br or I.

64. (canceled)

65. An isotopically encoded form of the water soluble substituted acetophenone of claim 61.

66. The isotopically encoded water soluble substituted acetophenone of claim 61, wherein X is any halogen, sulfonic ester, perchlorate ester or chlorosulfonate.

67. The isotopically encoded water soluble substituted acetophenone of claim 66, wherein
R₁ is SO₂H, at least one of, and preferably at least three of R₂-R₅ are ²H, and R₁′-R₆′ are ¹²C;
R₁ is SO₂H, R₂-R₅ are H, and at least one of, and preferably at least three of R₁′-R₆′ are ¹³C;
R₂ is SO₂H, at least one of, and preferably at least three of R₁ and R₃-R₅ are ²H, and R₁′-R₆′ are ¹²C;
R₂ is SO₂H, R₁ and R₃-R₅ are H, and at least one of, and preferably at least three of R₁′-R₆′ are ¹³C;
R₃ is SO₂H, at least one of, and preferably at least three of R₁-R₂ and R₄-R₅ are ²H, and R₁′-R₆′ are ¹²C;
R₃ is SO₂H, R₁-R₂ and R₄-R₅ are H, and at least one of, and preferably at least three of R₁′-R₆′ are ¹³C;
R₁ is SO₃H, at least one of, and preferably at least three of R₂-R₅ are ²H, and R₁′-R₆′ are ¹²C;
R₁ is SO₃H, R₂-R₅ are H, and at least one of, and preferably at least three of R₁′-R₆′ are ¹³C;
R₂ is SO₃H, at least one of, and preferably at least three of R₁ and R₃-R₅ are ²H, and R₁′-R₆′ are ¹²C;
R₂ is SO₃H, R₁ and R₃-R₅ are H, and at least one of, and preferably at least three of R₁′-R₆′ are ¹³C;
R₃ is SO₃H, at least one of, and preferably at least three of R₁-R₂ and R₄-R₅ are 2H, and R₁′-R₆′ are ¹²C;
R₃ is SO₃H, R₁-R₂ and R₄-R₅ are H, and at least one of, and preferably at least three of R₁′-R₆′ are ¹³C.

68. A reagent kit for analysis of amino acids, peptides or proteins by mass spectrometry comprising a container containing the substituted acetophenone of claim 56.

69. A reagent kit for analysis of amino acids, peptides or proteins by mass spectrometry comprising a container containing a substituted acetophenone, or a salt thereof, or a solvate thereof, having the following formula: 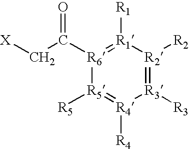
wherein X is a halide.

70. The reagent kit of claim 69, wherein X is Br or I.

71. The reagent kit of claim 69, wherein R₁-R₅ are H, and R₁′-R₆′ are ¹²C.

72. The reagent kit of claim 69, wherein the substituted acetophenone is an isotopically encoded substituted acetophenone, or a salt thereof, or a solvate thereof.

73. The reagent kit of claim 72, wherein at least one of, and preferably at least three of R₁-R₅ are ²H, and R₁ ′-R₆′ are ¹²C.

74. The reagent kit of claim 72, wherein R₁-R₅ are H, and at least one of, and preferably at least three of R₁′-R₆′ are ¹³C.

75. The reagent kit of claim 69, further comprising one or more containers containing: cysteine disulfide reducing agents, cysteine alkylating reagents, proteases or chemical cleavage agents, and solvents.

76. The reagent kit of claim 75, wherein the cysteine disulfide reducing agents are: dithiothreitol (DTT), β-mercaptoethanol, tris-carboxyethyl phosphine (TCEP), and/or tributylphosphine (TBP).

77. The reagent kit of claim 75, wherein the cysteine alkylating reagents are alkylhalides (e.g. iodoacetic acid, iodoacetamide), vinylpyridine or acrylamide.

78. The reagent kit of claim 75, wherein the proteases or chemical cleavage agents are trypsin, Endoproteinase Lys-C, Endoproteinase Asp-N, Endoproteinase Glu-C, pepsin, papain, thermolysin, cyanogen bromide, hydroxylamine hydrochloride, 2-[2′-nitrophenylsulfenyl]-3-methyl-3′-bromoindole (BNPS-skatole), iodosobenzoic acid, pentafluoropropionic acid and/or dilute hydrochloric acid.

79. The reagent kit of claim 75, wherein the solvents are urea, guanidine hydrochloride, acetonitrile, methanol and/or water.

80. An amino acid or peptide comprising an amino acid derivatized to include a side chain fixed-charge sulfonium ion, quaternary alkylammonium ion or quaternary alkylphosphonium ion.

81. (canceled)

82. The amino acid or peptide of claim 80, wherein the amino acid is derivatized using a substituted acetophenone, or a salt thereof, or a solvate thereof, having the following formula: 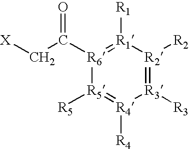
wherein X is a halide.

83. The amino acid or peptide of claim 82, wherein X is Br or I.

84. The amino acid or peptide of claim 82, wherein R₁-R₅ are H, and R₁′-R₆′are ¹²C or at least one of R₁′-R₆′ is ¹³C, or R₁-R₅ are ²H and R₁′-R₆′ are ¹²C.

85. The amino acid or peptide of claim 82, wherein the substituted acetophenone is an isotopically encoded substituted acetophenone, or a salt thereof, or a solvate thereof.

86-87. (canceled)

88. The amino acid or peptide of claim 80, wherein the amino acid derivative is isotopically encoded.

89. A method for providing an internal standard in a mass spectrometer method comprising adding to a sample a predetermined quantity of the fixed charge derivatized amino acid or peptide of claim 80.

90. A reagent kit for analysis of amino acids, peptides or proteins by mass spectrometry comprising a container containing the substituted acetophenone of claim 61.


SEQUENCE LISTING (12 sequences)

SEQ ID NO: 1 - protein, 10 aa, ARTIFICIAL SEQUENCE
  SYNTHETIC PEPTIDE
GAILMGAILA

SEQ ID NO: 2 - protein, 10 aa, ARTIFICIAL SEQUENCE
  SYNTHETIC PEPTIDE
GAILMGAILK

SEQ ID NO: 3 - protein, 10 aa, ARTIFICIAL SEQUENCE
  SYNTHETIC PEPTIDE
GAILMGAILR

SEQ ID NO: 4 - protein, 10 aa, ARTIFICIAL SEQUENCE
  SYNTHETIC PEPTIDE
GAILAGAILA

SEQ ID NO: 5 - protein, 10 aa, ARTIFICIAL SEQUENCE
  SYNTHETIC PEPTIDE
GAILAGAILK

SEQ ID NO: 6 - protein, 10 aa, ARTIFICIAL SEQUENCE
  SYNTHETIC PEPTIDE
GAILAGAILR

SEQ ID NO: 7 - protein, 10 aa, ARTIFICIAL SEQUENCE
  SYNTHETIC PEPTIDE
GAILXGAILX

SEQ ID NO: 8 - protein, 10 aa, ARTIFICIAL SEQUENCE
  SYNTHETIC PEPTIDE
GAILMGAILX

SEQ ID NO: 9 - protein, 5 aa, Bos taurus
IETMR

SEQ ID NO: 10 - protein, 12 aa, Bos taurus
ETYGDMADCCEK

SEQ ID NO: 11 - protein, 12 aa, Bos taurus
TVMENFVAFVDK

SEQ ID NO: 12 - protein, 14 aa, Bos taurus
MPCTEDYLSLILNR